(19) United States
(12) Patent Application Publication
(10) Pub. No.: US 2003/0203944 A1
(43) Pub. Date: Oct. 30, 2003

(54) Novel thiocarbamic acid derivatives and the pharmaceutical compositions containing the same

(76) Inventors: Young Ger Suh, Kyunggi-do (KR); Uh Taek Oh, Kyunggi-do (KR); Hee Doo Kim, Seoul (KR); Jee Woo Lee, Seoul (KR); Hyeung Geun Park, Seoul (KR); Young Ho Park, Seoul (KR); Jung Bum Yi, Kyunggi-do (KR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/343,703

(22) PCT Filed: Aug. 20, 2001
(86) PCT No.: PCT/KR01/01409

(30) Foreign Application Priority Data
Aug. 21, 2000 (KR) 2000-48387

(51) Int. Cl. A61K 31/44; A61K 31/4172; A61K 31/405; A61K 31/325; A61K 31/165; A61K 31/17

(52) U.S. Cl. 514/357; 514/400; 514/419; 514/485; 514/595; 514/580; 514/617; 546/331; 546/332; 548/338.1; 548/338.5; 548/503; 558/254; 560/24; 564/26; 564/48; 564/161

(57) ABSTRACT

The present invention relates to an antagonist against vanilloid receptor and the pharmaceutical compositions containing the same. As diseases associated with the activity of vanilloid receptor, pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fevescence, stomach-duodenal ulcer, inflammatory bowel disease and inflammatory diseases can be enumerated. The present invention provides a pharmaceutical composition for prevention or treatment of these diseases.

### TECHNICAL FIELD

The present invention relates to thiocarbamic acid derivatives and the pharmaceutical compositions containing the same, and particularly, to novel thiocarbamic acid derivatives as an antagonist against vanilloid receptor (VR) and the pharmaceutical compositions thereof.

### BACKGROUND ART

As diseases associated with the activity of vanilloid receptor, pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease and inflammatory diseases can be enumerated. The present invention provides pharmaceutical compositions for prevention or treatment of these diseases.

Yet, the diseases described above are only for enumeration, not to limit the scope of clinical application of vanilloid receptor antagonist.

Capsaicin (8-methyl-N-vanillyl-6-nonenamide) is a main pungent component in hot peppers. Hot peppers have been used, for a long time, not only as a spice but also as traditional medicine in the treatment of gastric disorders and when applied locally, for the relief of pain and inflammation (Szallasi and Blumberg, 1999, Pharm, Rev. 51, pp159-211). Capsaicin has a wide spectrum of biological actions, and not only exhibits effects on the cardiovascular and respiratory systems but also induces pain and irritancy on local application. Capsaicin, however, after such induction of pain, induces desensitization, both to capsaicin itself and also to other noxious stimuli to make the pain stopped. Based on this property, capsaicin and its analogues such as olvanil, nuvanil, DA-5018, SDZ-249482, resiniferatoxin are either used as analgesic agent, therapeutic agent for incontinentia urinae or skin disorder, or under development (Wriggleworth and Walpole, 1998, Drugs of the Future 23, pp 531-538).

Transmissions of mechanical, thermal and chemical noxious stimuli are mainly occurred by primary afferent nerve fibers of fine umnyelinated nerve (C-fiber) and thin myelinated nerve (A-fiber), and main reaction site of capsaicin and its analog called vanilloid is present at the nerve fiber transmitting the noxious stimuli. Capsaicin acts at the receptor existing on these neurons to induce potent stimuli by causing potent inflow of mono-and di-valent cations such as calcium and sodium, then exhibits potent analgesic effect by blocking the nervous function (Wood et al., 1988, J. Neurosci, 8, pp3208-3220). Vanilloid receptor (VR-1) has been recently cloned and its existence becomes clear (Caterina et al., 1997, Nature 389, pp816-824). It was clarified that this receptor transmits not only stimuli by capsaicin anlogues (vanilloid) but also various noxious stimuli such as proton and thermal stimuli (Tominaga et al., 1998, Neuron 21, pp531-543). Based on this, it is considered that vanilloid receptor functions as a integrative modulator against various noxious stimuli and carries out critical role in transmissions of pain and noxious stimuli. Recently, knock-out mouse in which gene encoding for vanilloid receptor was deleted was prepared (Caterina et al., 2000, Science 288, pp306-313; Davis et al., 2000, Nature 405, pp183-187). Compared to normal mice, the mouse was found out to exhibit much reduced reaction to thermal stimuli and thermal pain, while exhibiting no difference in general behavior, reconfirming the importance of the receptor in transmission of noxious signal. However, except proton, no other endogenous ligand, not exogenous ligand such as capsaicin, actually involved in transmission of noxious stimuli at vanilloid receptor was known. It is considered that leucotriene metabolite represented by 12-hydroperoxyeicosatetraenoic acid (12-IPETE) (Hwang et al., 2000, PNAS 11, pp6155-6160) and arachidonic acid derivatives such as anandamide (Zygmunt et al., 2000, Trends Pharmocol. Sci. 21, pp43-44) act as the most likely endogenous ligand for the receptor and proton acts as a cofactor with receptor-stimulating activity, rather than as a direct ligand.

As such, a capsaicin-sensitive sensory nerve cell and a vanilloid receptor existing in the cell are distributed over the entire body and play basic function in transmission of noxious stimuli and pain, further act as crucial factor in expression of neurogenic inflammation, thereby to have close relation with the cause of neuropathies, nerve injury, stroke, asthma, chronic obstructive pulmonary diseases, urinary bladder hypersensitiveness, irritable bowel syndrome, inflammatory bowel disease, fervescence, skin disorder and inflammatory disease. Lately, their correlation even with neuropathic disease is suggested (WO 99/00125). Recently, attention has focused to the role of afferent sensory nerve responding to capsaicin in gastrointestinal injury, and it was proposed that the afferent nerve might have a dual character that it exhibits protective action against gastric damage by improving gastric microcirculation through releasing peripheral neuropeptide such as CGRP (calcitonin gene-related peptide), while inducing gastric injury by stimulating sympathetic nervous system (Ren et al., 2000, Dig. Dis. Sci. 45, pp830-836). It is determined that vanilloid receptor antagonist has very high potential to be used for prevention or treatment of the said various diseases by blocking the vanilloid receptor conducting such varied functions.

Though it may be, theoretically, anticipated that antagonist for this receptor would exhibit substantial degree of inhibitory action against pain and neurogenic inflammation, it was found out that the competitive antagonist for this receptor, capsazepine, almost the only one known until now, failed to exhibit significant analgesic and anti-inflammatory effects (Perkins and Campbell, 1992, Br. J. Pharmacol. 107, pp329-333). Therefore, not much progress was made on this field. However, recently, there has been a report on significant results for analgesic action of capsazepine in animal studies (Kwak et al., 1998, Neurosci. 86, pp619-626; Santos and calixto, 1997, Neurosci. Lett. 235, pp73-76), in particular, the inventors of the present invention clearly demonstrated through animal studies the analgesic and anti-inflammatory effects of the strong vanilloid receptor antagonists which were identified through experiments in laboratory, and based on this, strongly suggested the development potential of vanilloid receptor antagonist as an analgesic and anti-inflammatory agent. Yet, though the vanilloid receptor antagonist derived from the present studies will mainly act based on the antagonistic activity of itself, even a possibility that it could exhibit the pharmacological activity through transformation into agonist via metabolism after absorption into body is not to be excluded.

To resolve the problems described above, the present invention is to provide novel compounds which are selectively antagonistic to vanilloid receptor and exhibit analgesic and anti-inflammatory effects while causing no irritancy, and pharmaceutical compositions containing the same.

### DISCLOSURE OF THE INVENTION

In order to attain the above objects, the present invention provides a novel compound of formula (I):

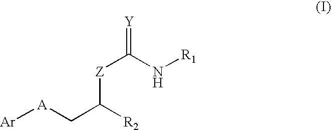

wherein,

R₁ represents Ar′—(CH₂)ₘ— (wherein Ar′ is phenyl, pyridinyl, thiophenyl or naphthalenyl substituted or unsubstituted with halogen or lower alkyl having 1 to 5 carbon atoms; or trifluoromethylphenyl, and m is 1, 2, 3 or 4), —(CH₂)ₙ—CHPh₂, or —CH₂CH₂CH(Ph)CH₂Ph (wherein n is 1 or 2);

Y represents S or O;

Z represents O, —CH₂—, NR₃, CHR₃ (wherein R₃ is hydrogen, lower alkyl having 1 to 5 carbon atoms, benzyl or phenethyl);

R₂ represents hydrogen, lower alkyl having 1 to 6 carbon atoms, cycloalkyl, dimethyl, or Ar″—(CH₂)ₚ— (wherein Ar″ is phenyl substituted or unsubstituted with halogen or trifluoromethyl; or pyridinyl, imidazolyl or indolyl substituted or unsubstituted with carboxyl, amino, methanesulfonylamino or t-butoxycarbonyl, p is 0, 1, 2, 3 or 4.);

A represents O or —CH₂—; and

Ar represents

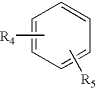

(wherein R₄ and R₅ each independently are hydrogen, hydroxy, methoxy, nitro, cyano, benzyloxy, amino, methanesulfonylamino, halogen, lower alkyl having 1 to 5 carbon atoms, —NHCO₂CH₃, —NHC(═O)CH₃, trifluoromethyl, sulfamoyl, carboxyl, —OCH₂OCH₃, methoxycarbonyl); or pyridinyl, indolyl or imidazolyl substitituted or unsubstituted with carboxyl, amino, methanesulfonylamino, phenethylaminocarbonyl or t-butoxycarbonyl.

The present invention also provides a pharmaceutical composition comprising a compound of formula (I) or a pharmaceutically acceptable salt thereof as an active ingredient.

The compounds according to the present invention can be synthesized chemically by the following reaction schemes. However, these are given only for illustration of the invention and are not intended to limit to them.

First, the below compound 6, within the scope of the compound (I) according to the present invention, is synthesized by the following Scheme 1.

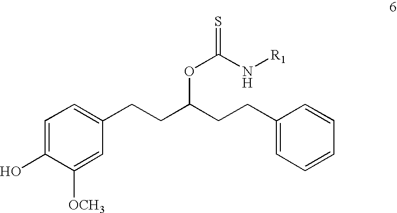

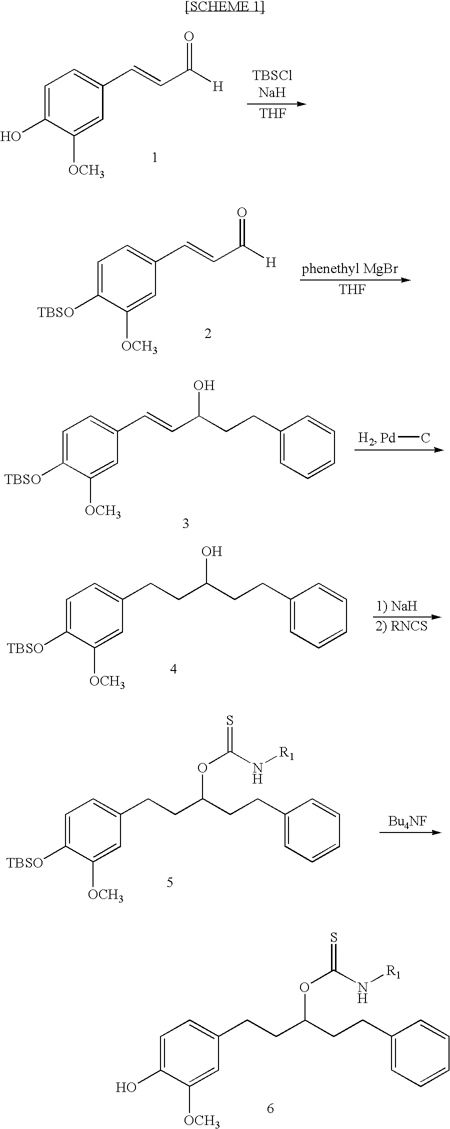

Referring to the above Scheme 1, aryl alcohol compound 3 is obtained by protecting hydroxy group of cinnamaldehyde compound 1 with silyl group and then by reacting phenethyl magnesium bromide therewith. Double bond of compound 3 is subjected to catalytic hydrogenation thereby to obtain alcohol. Alkoxide is prepared from the alcohol using sodium hydride, and then reacted with one of various kinds of alkyl, arylalkyl and aryl isothiocyanate to synthesize thiocarbamate compound 5. The protecting group is removed therefrom to obtain the title compound 6 within the scope of the compound (I) according to the present invention.

Next, the below compound 10, within the scope of the compound (I) according to the present invention, is synthesized by the following Scheme 2.

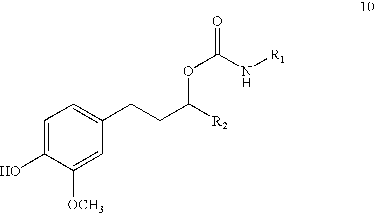

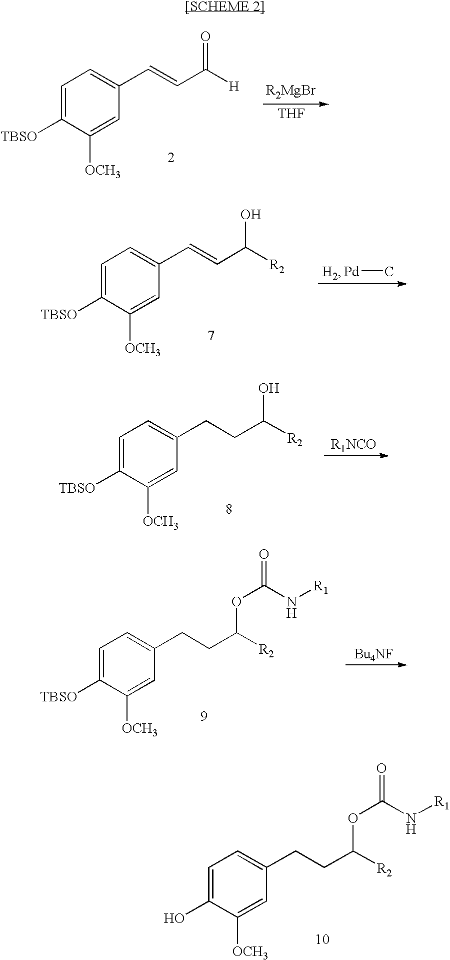

Referring to the above Scheme 2, cinnamaldeyde compound 2 is reacted with Grignard reagent to prepare aryl alcohol compound 7, and double bond of compound 7 is subjected to catalytic hydrogenation. Alcohol group of the reduced compound is reacted with isocyanate group to obtain carbamate 9, and the protecting group is removed therefrom to obtain carbamate compound 10 within the scope of the compound (I) according to the present invention.

Next, the below compound 14, within the scope of the compound (I) according to the present invention, is synthesized by the following Scheme 3.

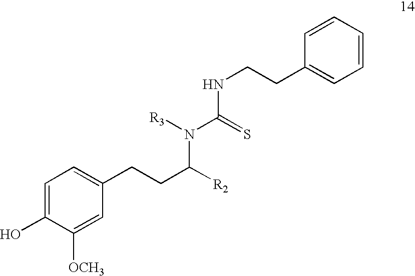

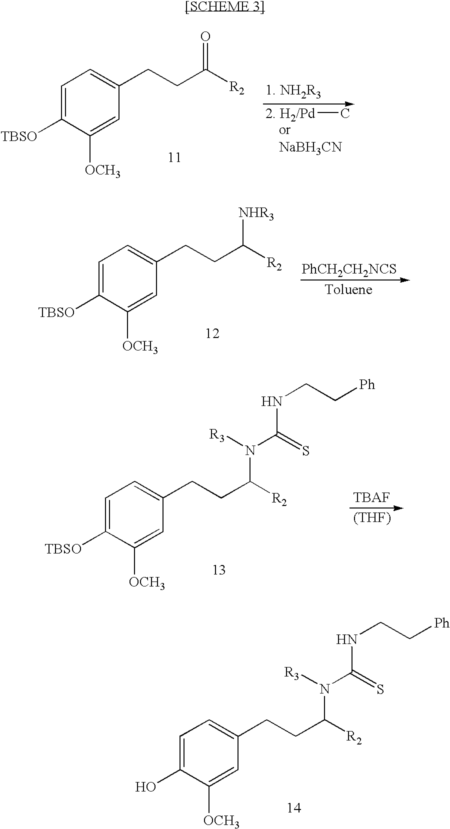

Referring to the above Scheme 3, ketone compound 11 undergoes reductive amination with alkyl amine, benzyl amine and the like, by which compound 11 is first converted to imine and then the imine is converted to compound 12. Phenethyl isothiocyanate is reacted therewith to obtain thiourea compound 13, and then the protecting group is removed therefrom to obtain compound 14 within the scope of the compound (I) according to the present invention.

Next, the below compound 16, within the scope of the compound (I) according to the present invention, is synthesized by the following the Scheme 4.

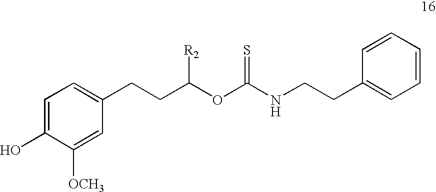

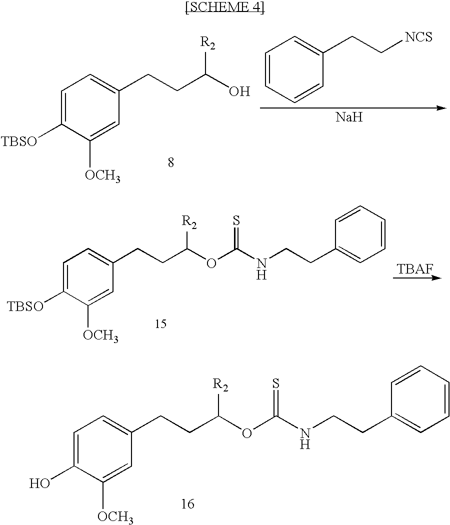

Referring to the above Scheme 4, alkoxide is prepared by reaction of sodium hydride with alcohol compound 8 in which R₂ are structurally various, to prepare the corresponding alkoxide, and then reacted with phenethyl isothiocyante to synthesize isothiocarbamate compound 15. The protecting group is removed therefrom to obtain compound 16 within the scope of the compound (I) according to the present invention.

Next, the below compound 25, within the scope of the compound (I) according to the present invention, is synthesized by the following Scheme 5.

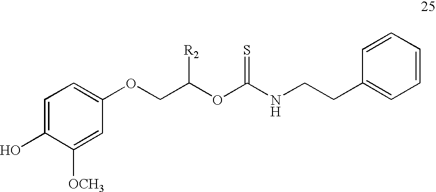

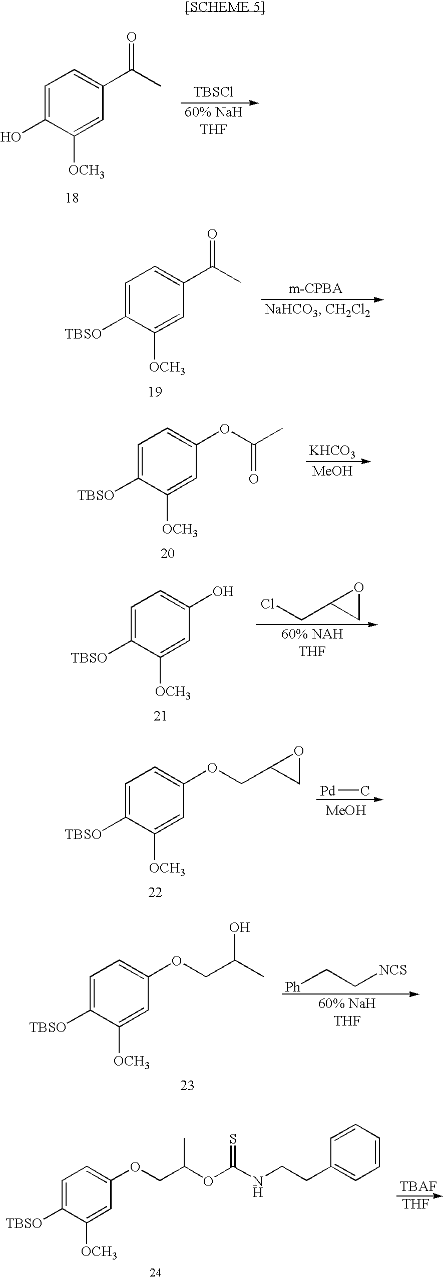

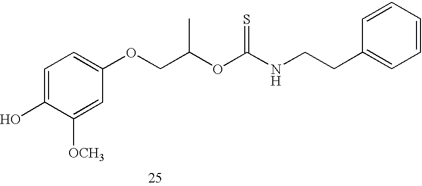

Referring to the above Scheme 5, acetovanillone compound 18 is protected with TBS and then allowed to Bayer-Villiger oxidative reaction, that is, oxidation with m-CPBA, to obtain ester compound 20. The compound 20 is hydrolyzed to obtain phenol, and epichlorohydrin is reacted therewith to obtain epoxy ether compound 22. The obtained compound 22 is subjected to contact catalytic reduction to obtain alcohol compound 23, and phenethyl isocyanate is reacted therewith to obtain isothiocarbamate. The protecting group is removed therefrom to obtain compound 25 within the scope of the compound (I) according to the present invention.

Next, the below compound 28, within the scope of the compound (I) according to the present invention, is synthesized by the following Scheme 6.

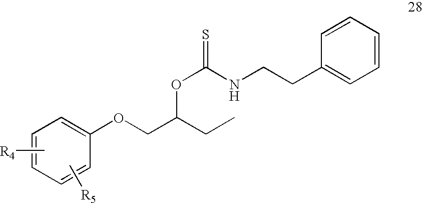

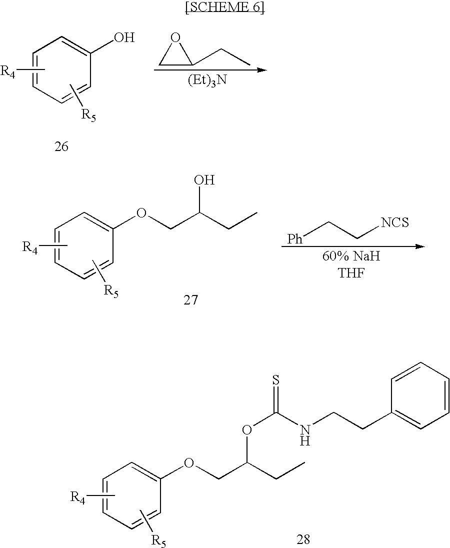

Referring to the above Scheme 6, phenol-based compound 26 is reacted with epoxy butane in the presence of base to obtain compound 27. Alcohol group of compound 27 is reacted with isothiocyanate to obtain isothiocarbamate compound 28 within the scope of the compound (I) according to the present invention.

Next, the below compound 30 or 31, within the scope of the compound (I) according to the present invention, is synthesized by the following Scheme 7.

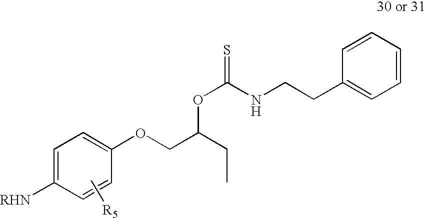

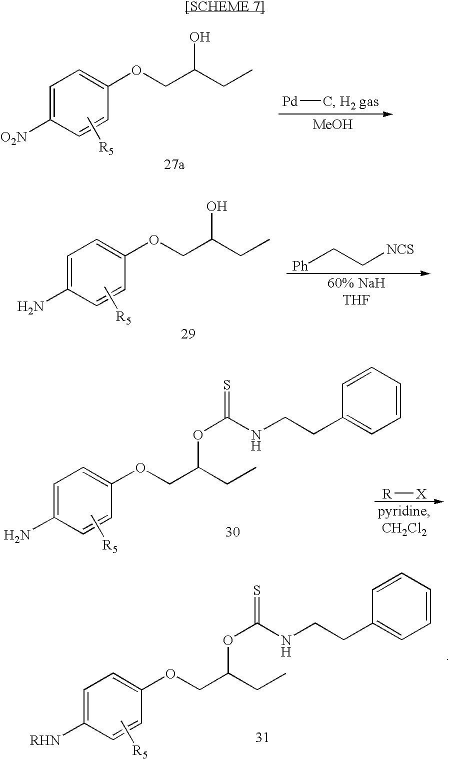

Referring to the above Scheme 7, compound 27a undergoes catalytic hydrogenation to yield aminoalcohol compound 29 and alcohol group of compound 29 is selectively reacted with phenethyl isothiocyanate to obtain phenethyl thiocarbamate compound 30 within the scope of the compound (I) according to the present invention. And, the obtained compound 30 is reacted with benzoyl chloride, acetic anhydride, methanesulfonic anhydride, methyl chloroformate and the like to obtain compound 31 within the scope of the compound (I) according to the present invention Next, the below compound 37, within the scope of the compound (I) according to the present invention, is synthesized by the following Scheme 8.

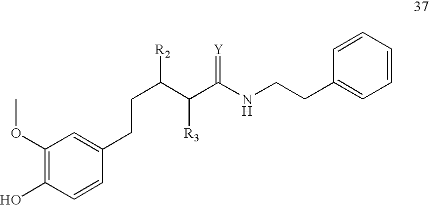

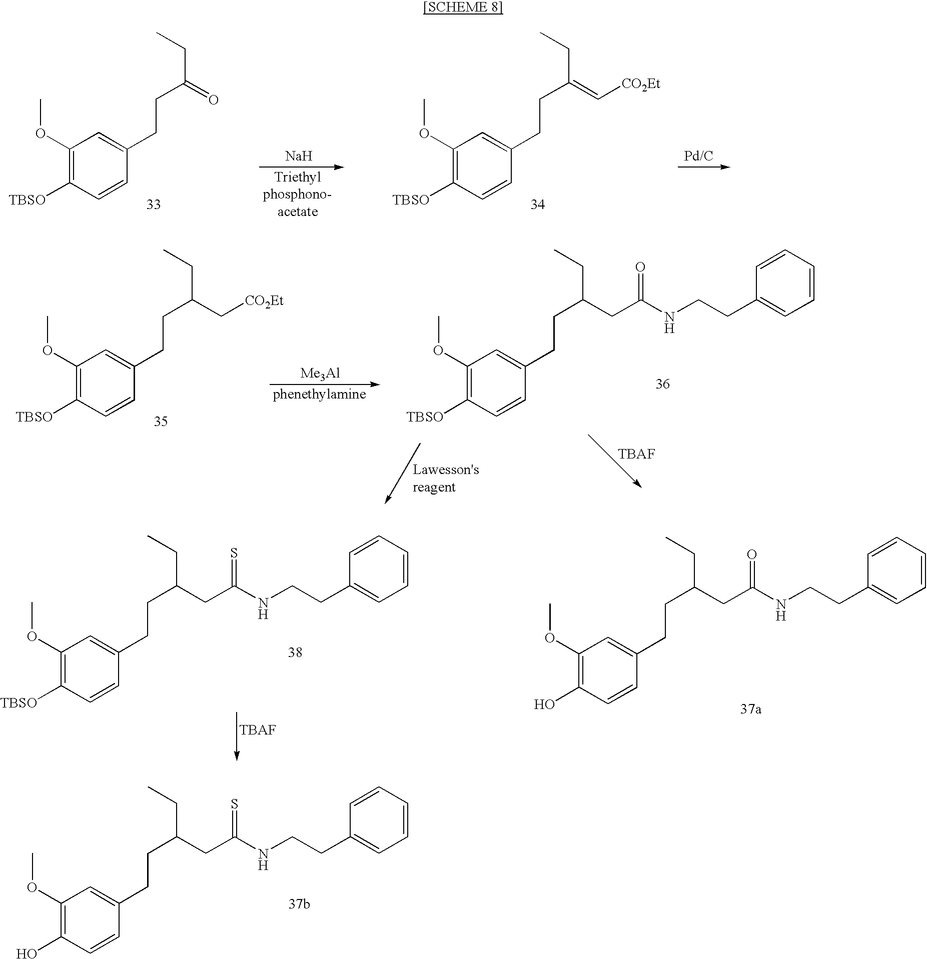

Referring to the above Scheme 8, ketone compound 33 is reacted with trialkyl phosphonoalkanoate to synthesize α,β-unsaturated ester. Double bond of the ester is subjected to catalytic hydrogenation, and the reduced ester is converted to amide in the presence of trimethyl aluminum as a catalyst. Compound 37a, within the scope of the compound (I) according to the present invention, is obtained by removing the protecting group from the synthesized amide. Alternatively, thioamide compound 37b, within the scope of the compound (I) according to the present invention, is obtained by reacting the synthesized amide with lawesson's reagent to prepare compound 38 and then removing the protecting group therefrom.

Next, the below compound 39, within the scope of the compound (I) according to the present invention, is synthesized by the following Scheme 9.

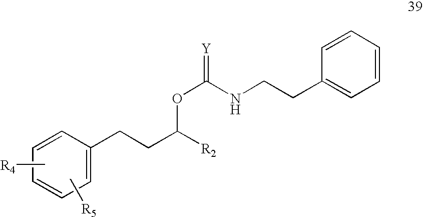

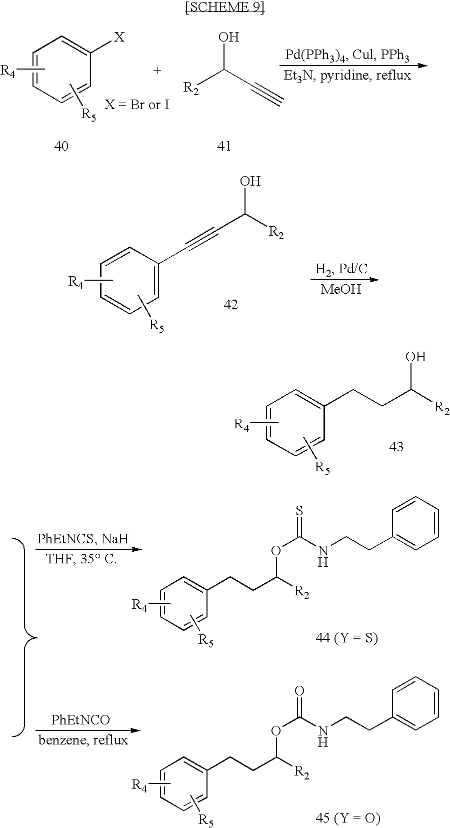

Referring to the above Scheme 9, halobenzene compound 40 substituted with R₄ and R₅ is bonded to phenethyl propargyl alcohol 41 in the presence of palladium catalyst. Triple bond of intermediate compound 42 is reduced to obtain compound 43, followed by reacting phenethyl isothiocyanate and the like therewith to synthesize compounds 44 and 45 within the scope of the compound 39.

Carboxylic acid is obtained from 3-bromophenol using carbon tetrachloride, sodium hyroxide and the like, and then treated with diazomethane to obtain ester 40c-1 (R₄=4-methoxycarbonyl, R₅=3-hydroxy). Hydroxyl group is protected with a methoxymethyl group to obtain compound 40c-2 and then compound 43c-1 (R₄=4-methoxycarbonyl, R₅=3-methyloxymethoxy) is obtained therefrom in accordance with the above Scheme 9. Compound 43c-1 is hydrolyzed to obtain compound 43c-2 (R₄=4-carboxyl, R₅=3-methyloxymethoxy), and the phenethyl isocyanate is reacted therewith to synthesize compound 44c-1. Then the trifluoroacetic acid is reacted therewith to obtain compound 44c-2 (R₄=4-carboxyl, R₅=3-hydroxy).

4-Bromo-o-xylene is oxidized with potassium permanganate to obtain benzoic acid and diazomethane is reacted therewith to synthesize compound 36. Compound 36 is reacted in accordance with the above Scheme 5 to obtain dibenzoic acid compound 40.

Next, the below compounds 46, 51 and 53, within the scope of the compound (I) according to the present invention, are synthesized by the above Scheme 9 or the following Scheme 10.

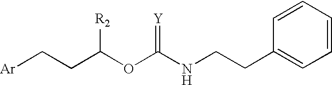

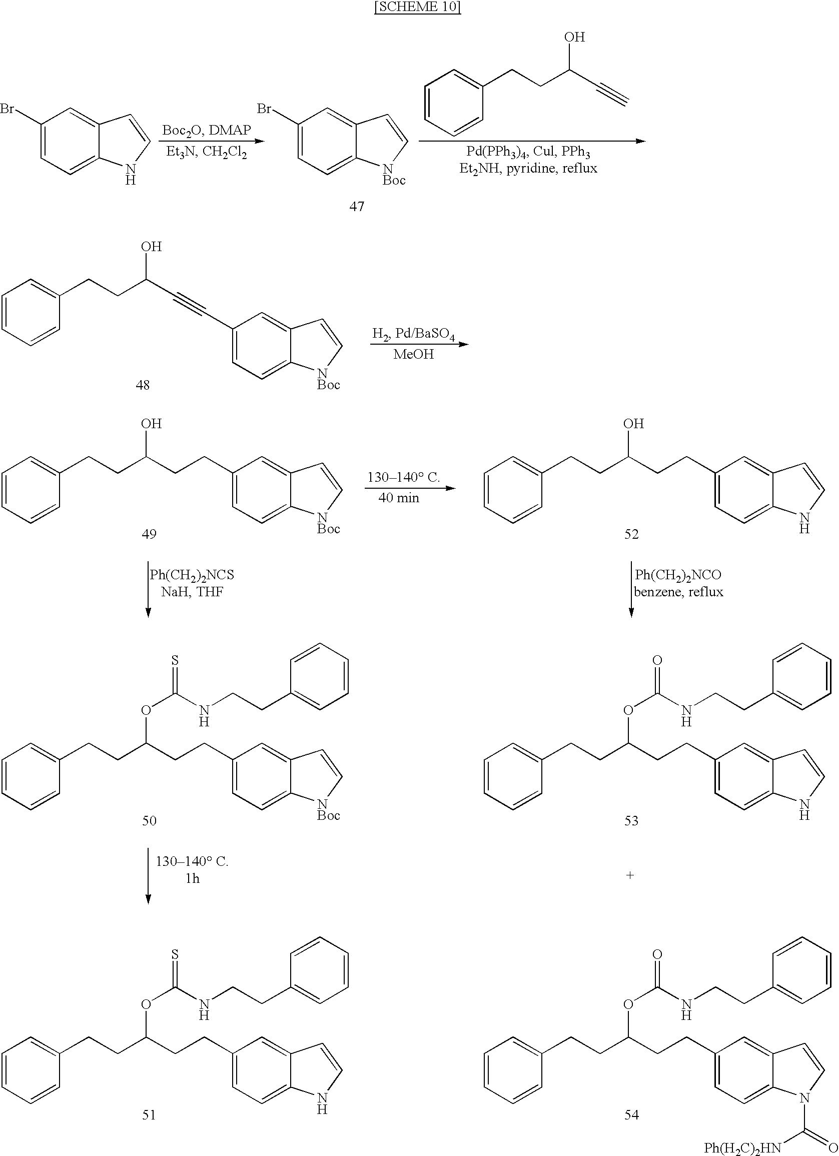

Pyridine derivatives 46a˜e of compound 46 are synthesized in accordance with the above Scheme 9, and imidazole derivatives 46f and indole derivatives 51 and 53 are synthesized in accordance with the above Scheme 10. Referring to the above Scheme 10, 5-bromoindole is protected with butyloxycarbonyl group and reacted with 5-phenyl-1-pentyn-3-ol compound in the presence of palladium catalyst to compound 48. Triple bond of compound 48 undergoes catalytic hydrogenation to yield compound 49, and then its butyloxycarbonyl is deprotected to obtain compound 52. Compounds 51 and 53 are obtained, in accordance with the above Scheme 10, using compounds 49 and 52 as starting material, respectively.

Next, the below compound 60, within the scope of the compound (I) according to the present invention, is synthesized by the following Scheme 11.

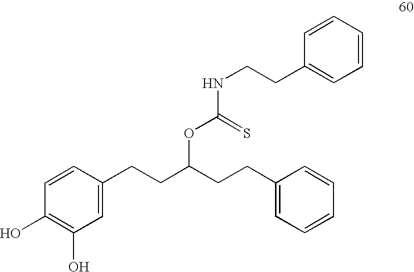

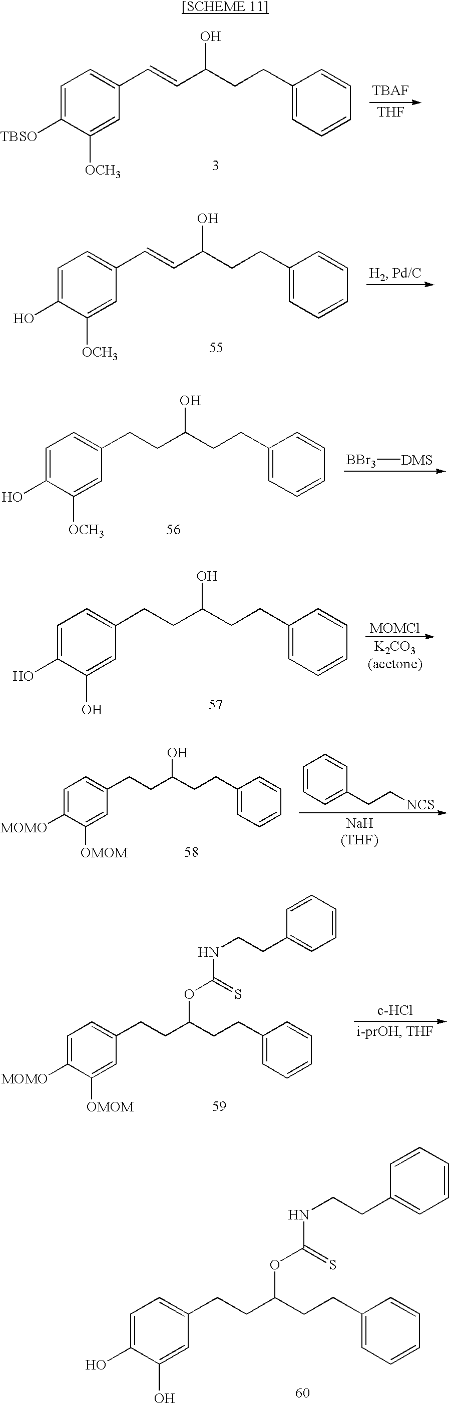

Referring to the above Scheme 11, in succession, TBS group is removed from compound 3, followed by reducing double bond thereof and then removing methyl group thereof, to obtain compound 57. Phenol group whose acidity is high is selectively protected with potassium carbonate, and alcohol group at the other position is reacted with isothiocyanate to obtain thiocarbamate. The protecting group is removed therefrom using hydrochloric acid to obtain compound 60.

The compound of formula (I) according to the present invention can be provided as a pharmaceutical composition containing pharmaceutically acceptable carriers, adjuvants, or diluents. For instance, the compounds of the present invention can be dissolved in oils, propylene glycol or other solvents which are commonly used to produce an injection. Suitable examples of the carriers include, physiological saline, polyethylene glycol, ethanol, vegetable oils, isopropyl myristate, etc., but are not limited to them. For topical administration, the compounds of the present invention can be formulated in the form of ointments and creams.

The pharmaceutical composition containing the compound of the present invention as an active ingredient can be used for treating acute, chronic, inflammatory or neuropathic pains; treating urinary bladder hypersensitiveness or irritable bowel syndrome (EBS); treating asthma; preventing or treating neurodegenerative diseases; or preventing or treating neurotic skin disorder, or irritation of skin, eye or mucous membrane.

Hereinafter, the formulating methods and kinds of excipients will be described, but the present invention is not limited to them.

The compound according to the present invention may also be used in the forms of pharmaceutically acceptable salts thereof, and may be used either alone or in combination or in admixture with other pharmaceutically active compounds.

The compounds of the present invention may be formulated into injections by dissolving, suspending or emulsifying in water-soluble solvent such as saline and 5% dextrose, or in water-insoluble solvents such as vegetable oils, synthetic fatty acid glyceride, higher fatty acid esters and propylene glycol. The formulations of the invention may include any of conventional additives such as dissolving agents, isotonic agents, suspending agents, emulsifiers, stabilizers and preservatives.

The preferable dose level of the compounds according to the present invention depends upon a variety of factors including the condition and body weight of the patient, severity of the particular disease, dosage form, and route and period of administration, but may appropriately be chosen by those skilled in the art. The compounds of the present invention are preferably administered in an amount ranging from 0.001 to 100 mg/kg of body weight per day, and more preferably from 0.01 to 30 mg/kg of body weight per day. Doses are administered once a day or several times a day with devided portions. The compounds of the present invention must be present in a pharmaceutical composition in an amount of 0.0001˜10% by weight, and preferably 0.001˜1% by weight, based on the total amount of the composition.

The pharmaceutical composition of the present invention can be administered to a mammalian subject such as rat, mouse, domestic animals, human being and the like via various routes. The methods of administration which may easily be expected include oral and rectal administration; intravenous, intramuscular, subcutaneous, intrauterine, duramatral and intracerebroventricular injections.

### MODELS FOR CARRYING OUT THE INVENTION

The present invention is more specifically explained by the following examples. However, it should be understood that the present invention is not limited to these examples in any manner.

### EXAMPLE 1

### Synthesis of 4-(t-butyldimethylsilyloxy)-3-methoxy Cinnamaldehyde (2)

Cinnamaldehyde 1 (1.71 g, 9.6 mmol) was diluted in tetrahydrofuran (15 ml), and then sodium hydride (60%, 1.15 g, 28.7 mmol) was added thereto. The resulting mixture was stirred for 30 minutes. The mixture was cooled to 0° C., and a solution of t-butyldimethylsilyl chloride in THF (5 ml) was slowly added thereto, followed by stirring for 7 hours. After the completion of the reaction was confirmed using TLC, saturated aqueous ammonium chloride solution was added thereto to quench the reaction. The reaction mixture was extracted with ethyl acetate (100 ml). The organic layer was washed with saturated aqueous ammonium chloride solution (15 ml), water (15 ml×3) and saturated aqueous sodium chloride solution (15 ml), and then dired over anhydrous sodium sulfate. The reaction mixture was concentrated under reduced pressure, and the obtained residue was column-chromatographed (n-hexane/ethyl acetate—20/1) to yield the compound 2 (27.8 g, 99.3%) as a pale yellow crystal.

IR (KBr) 3430, 3007, 2857, 2748, 1674, 1621, 1596, 1511, 1465, 1288, 1128 cm⁻¹; ¹H NMR (300 MHz, CDCl₃): 9.47(1H, d, J=7.7 Hz), 7.22(1H, d, J=15.8 Hz), 6.89(2H, m), 6.70(1H, d, J=7.9 Hz), 6.42(1H, dd, J=15.8 Hz, 7.7 Hz), 3.67(3H, s), 0.82(9H, s), 0.00(6H, s).

### EXAMPLE 2

### Synthesis of 4-[(E)-3-(t-butyldimethylsilyloxy)-5-phenylpent-1-enyl]-2-methoxyphenol (3)

Compound 2 (550 ma, 1.88 mmol) was diluted in tetrahydrofuran (8 ml), and then the diluted solution was cooled to −78° C. Phenethyl magnesium bromide (1M solution, 2.8 ml, 2.82 mmol) was slowly added thereto and the mixture was stirred for 30 minutes and then cooled to room temperature. After completion of the reaction was confirmed using TLC, saturated aqueous ammonium chloride solution was added thereto to terminate the reaction. The reaction mixture was extracted with ethyl acetate (50 ml). The organic layer was washed successively with saturated aqueous ammonium chloride solution (8 ml), water (8 ml×3) and saturated aqueous sodium chloride solution (8 ml), and then dried over anhydrous sodium sulfate. The reaction mixture was concentrated under reduced pressure, and the obtained residue was column-chromatographed (n-hexane/ethyl acetate=15/1, SiO₂) to yield the compound 3 (740 mg, 99.4%) as a pale yellow oil.

IR (neat) 3353, 3026, 2929, 2857, 1601, 1512, 1464, 1281 cm⁻¹; ¹H NMR (300 MHz, CDCl₃): 7.17-7.01(5H, m), 6.74(1H, d, J=1.8), 6.69(1H, dd, J=8.1, 1.8), 6.64(1H, d, J=8.1), 6.35(1H, d, J=15.7), 5.95(1H, dd, J=15.9, 7.0), 4.21(1H), 3.67(3H, s), 2.69-2.53(2H, m), 1.86-1.75(2H, m), 1.43-1.40(1H, m), 0.84(9H, s), 0.00(611, s)

### EXAMPLE 3

### Synthesis of 4-[3-(t-butyldimethylsilyloxy)-5-phexiylpentyl]-2-methoxyphenol (4)

Compound 3 (176 mg, 0.44 mmol) was diluted in ethanol, and palladium/carbon (30 mg) was added thereto, followed by filling the inside of flask with hydrogen gas and then sting. After the completion of the reaction was confirmed using TLC, the reaction mixture was filtered to remove palladium/carbon and the filtrate was concentrated under reduced pressure. The obtained residue was column-chromatographed (n-hexane/ethyl acetate—4/1) to yield the compound 4 (145 mg, 82.3%) as a colorless oil.

IR (neat) 3374, 3027 cm⁻¹; ¹H NMR (300 Hz, CDCl₃) 7.18-7.04(5H, m), 6.61(1H, d, J=7.9), 6.53(1H, d, J=1.9), 6.48(1H, dd, J=7.9, 1.9), 3.64(3H, s), 3.56-3.48(1H, m), 2.70-2.40(4H, m), 1.70-1.61(4H, m), 1.32(1H, s), 0.85(9H, s), 0.00(6H, s)

### EXAMPLE 4

### Synthesis of Phenethylthiocarbamic Acid O-3-[4-(t-butyldimethylsitanyl)-3-methoxyphenyl]-1-phenethylproyl Ester (5a) (R₁=CH₂CH₂Ph)

Compound 4 (157 mg, 0.39 mmol) was added, through cannular, to tetrahydrofuran (5 ml) to obtain a diluted solution, and after adding 60% NaH in oil (47 mg, 1.17 mmol) thereto, the mixture was stirred at 30° C. for 1 hour. Phenethyl isothiocyanate (0.2 ml, 1.37 mmol) was slowly added thereto, followed by stirring for 24 hours. After completion of the reaction was confirmed using TLC, saturated aqueous ammonium chloride solution was added thereto to terminate the reaction. The reaction mixture was extracted with ethyl acetate (60 ml). The organic layer was washed with saturated aqueous ammonium chloride solution (7 ml), water (7 ml×3) and saturated aqueous sodium chloride solution (7 ml), and then dried over anhydrous Na₂SO₄. The reaction mixture was concentrated under reduced pressure, and the obtained residue was column-chromatographed (n-hexane/ethyl acetate=50/1, SiO₂) to yield the compound 5a (200 mg, 90.9%) as a colorless oil.

IR (neat) 3363, 3027, 2930, 2857, 1734, 1604, 1584, 1514, 1454, 1398, 1279, 1253, 1233 cm⁻¹; ¹H NMR (300 MHz, CDCl₃) 7.18-7.03(10H, m), 6.60(1H, d, J=8.0), 6.53(1H, d, J32.0), 6.46(1H, dd, J=8.0, 2.0), 5.96(1H, t, J=5.9), 5.52-5.41(1H, m), 3.68-3.64(4H, m), 3.31(1H, q, J=6.7), 2.80(1H, t, J=7.0), 2.68(1H, t, J=7.2), 2.53-2.45(4H, m), 1.86-1.79(4H, m), 0.85(9H, s), 0.00(6H, s).

### EXAMPLE 5

### Synthesis of Phenethyl Thiocarbamic Acid O-[3-(4-hydroxy-3-methoxyphenyl)-1-phenethylpropyl] Ester (6a) (R₁=PhCH₂CH₂)

Compound 5a (168 mg, 0.30 mmol) was dissolved in tetrahydrofuran (6 ml), and tetrabutylammonium fluoride (1M solution, 0.75 ml, 0.75 mmol) was slowly added. After stirring the mixture for 20 minutes, the completion of the reaction was confirmed using TLC. The reaction mixture was extracted with ethyl acetate. The organic layer was washed successively with water (4 ml×2) and saturated aqueous sodium chloride solution (4 ml), and then dried over Na₂SO₄. The resulting mixture was concentrated under reduced pressure and the obtained residue was column-chromatographed (n-hexane/ethyl acetate=10/1) to yield the pure compound (128 mg, 94.5%), as a colorless oil.

IR (neat) 3526, 3363, 3026, 2947, 1604, 1515, 1453, 1400, 1270, 1233 cm−1; ¹H NMR (300 MHz CDCl₃) 7.24-7.09(10H, m), 6.75(1H, d, J=8.0), 6.63-6.57(2H, m), 6.04(1H, t, 3=5.7), 5.58-5.48(1H, m), 5.40(1H, s), 3.80-3.70(4H, m), 3.37(1H, q, J=6.8), 2.86(1H, t, J=7.0), 2.74(1H, t, J=7.2), 2.62-2.48(4H, m), 1.99=1.83(4H, m); MS (EI) m/e(relative intensity) 449(M⁺) 416(2) 268(100) 137(73) 91(28)

Compounds 6b˜t were synthesized according to the similar procedure as synthesizing method of the compound 6a, and parts of spectral data thereof are shown below.

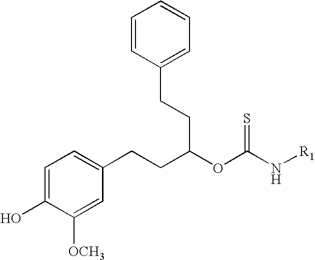

| Examp | Comp | | |
|---|---|---|---|
| les | ounds | R₁ | Spectral data |
| 6 | 6b | 3-phenyl- | ¹H NMR(300MHz, CDCl₃) 7.31-7.14 |
| | | propyl | (10H, m), 6.82(1H, s, J=8.0), 6.70- |
| | | | 6.62(2H, m), 6.55(2/5H, t, J=5.8), |
| | | | 6.03(3/5H, t, J=5.8), 5.60-5.54(1H, |
| | | | m), 5.45(1H, d, J=3.0), 3.86(3H, s), |
| | | | 3.57(6/5H, dd, J=2.8, 7.4), 3.20(4/5H, |
| | | | dd, J=13.1, 7.0), 2.72-2.56(6H, m), |
| | | | 2.10-1.75(6H, m) |
| 7 | 6c | 4-phenylbut | ¹H NMR(300MHz, CDCl₃) 7.32-7.14 |
| | | yl | (10H, m), 6.82(1H, d, J=8.1), 6.69 |
| | | | (1H, d, J=1.8), 6.65(1H, dd, J=8.1, |
| | | | 1.8), 6.51(2/5H, t, J=5.2), 6.03(3/5H, |
| | | | t, J=5.4), 5.66-5.51(1H, m), 5.44 |
| | | | (1H, d, J=2.6), 3.86(3H s), 3.55(6/5H, |
| | | | dd, J=12.6, 7.0), 3.18(4/5H, dd, |
| | | | J=12.7, 6.7), 2.70-2.56(6H, m), |
| | | | 2.10-1.86(4H, m), 1.74-1.54(4H, m) |
| 8 | 6d | 2-(4-chlorop | ¹H NMR(300MHz, CDCl₃) 7.22-7.01 |
| | | henyl)ethyl | (9H, m), 6.75(1H, d, J=8.0), 6.62(1H, |
| | | | d, J=2.0), 6.58(1H, dd, J=8.0, 2.0), |
| | | | 6.44(1/3H, t, J=5.5), 6.00(2/3H, t, |
| | | | J=5.9), 5.55-5.47(1H, m), 5.39(1H, |
| | | | s), 3.81(2H, s), 3.80(1H, s), 3.70(4/3H, |
| | | | dd, J=13.3, 6.3), 3.32(2/3H, dd, |
| | | | J=13.7, 6.6), 2.84(4/3H, t, J=7.0), |
| | | | 2.70(2/3H, t, J=7.1), 2.64-2.49(4H, |
| | | | m), 2.03-1.75(4H, m) |
| 9 | 6e | 2-(4-methylp | ¹H NMR(300Mhz, CDCl₃) 7.31-7.04 |
| | | henyl)ethyl | (9H, m), 6.82(1h, d, J=7.8), 6.70 |
| | | | (1H, d, J=1.8), 6.65(1H, dd, J=7.8, |
| | | | 1.8), 6.54(1/3H, t, J=5.6), 6.10(2/3H, |
| | | | t, J=5.9), 5.64-5.53(1H, m), 5.46 |
| | | | (1H, s), 3.87(2H, s), 3.86(1H, s), 3.79 |
| | | | (4/3H, dd, J=12.8, 6.8), 3.42(2/3H, dd, |
| | | | J=12.8, 7.0), 2.89(4/3H, t, J=6.9), |
| | | | 2.77(2/3H, t, J=7.2), 2.72-2.55(4H, |
| | | | m), 2.32(2H, s), 2.30(1H, s), 2.05-1.85 |
| | | | (4H, m) |
| 10 | 6f | 2-(4-fluorop | ¹NMR(300MHz, CDCl₃) 7.32-6.96 |
| | | henyl)ethyl | (9H, m), 6.84(1H, d, J=8.0), 6.71-6.65 |
| | | | (2H, m), 6.54(1/3H, t, J=6.0), 6.09 |
| | | | (2/3H, t, J=6.0), 5.65-5.57(1H, m), |
| | | | 5.48(1H, s), 3.89(2H, s), 3.88(1H, s), |
| | | | 3.79(4/3H, dd, J=13.9, 6.9), 3.41 |
| | | | (2/3H, dd, J=13.5, 6.5), 2.92(4/3H, |
| | | | t, J=7.5), 2.78(2/3H, t, J=7.2), |
| | | | 2.69-2.56(4H, m), 2.19-1.87(4H, m) |
| 11 | 6g | 2-(3,4-dichlo | ¹H NMR(300MHz, CDCl₃) 7.37-7.09 |
| | | rophenyl)eth | (8H, m), 6.81(1H, d, J=7.9), 6.68(1H, |
| | | | d, J=1.9), 6.64(1H, dd, J=7.9, 1.9), |
| | | | 6.57(1/3H, t, J=5.8), 6.12(2/3H, t, |
| | | | J=6.1), 5.56(1H, m), 5.45(1H, s), |
| | | | 3.86(2H, s), 3.86(1H, s), 3.78(4/3H, |
| | | | dd, J=13.4, 6.5), 3.41(2/3H, dd, |
| | | | J=14.0, 6.5), 3.05(4/3H, t, J=7.0), |
| | | | 2.90(2/3H, t, J=7.0), 2.69-2.54 |
| | | | (4H, m), 2.11-1.85(4H, m) |
| 12 | 6h | 2-(2,4-di- | ¹H NMR(300MHz, CDCl₃) 7.32-7.08 |
| | | chloro- | (15H, m) 6.80(1/3H, d, J=8.0) 6.78 |
| | | phenyl)ehtyl | (2/3H, d, J=8.0) 6.67-6.58(2H, m) |
| | | | 6.41(1/3H, t, J=5.3) 6.04(2/3H, t, |
| | | | J=5.3) 5.60-5.5.1(1H, m) 5.43 |
| | | | (1H, s) 4.38(2/3H, t, J=8.0) 4.18- |
| | | | 4.07(5/3H, m) 3.84(2H, s) 3.82(1H, s) |
| | | | 3.78-3.74(2/3H, m) 2.68-2.44(4H, m) |
| | | | 2.01-1.83 (4H, m) |
| 13 | 6i | 2,2-di- | ¹H NMR(300MHz, CDCl₃) 7.43-7.289 |
| | | phenyl- | (15H, m) 6.97(3/5H, d, J=7.9) 6.96 |
| | | ethyl | (2/3H, d, J=8.0) 6.84-6.75(12/5H, m) |
| | | | 6.15(3/5H, t, J=5.7) 5.77-5.60(1H, m) |
| | | | 5.62(2/5H, s) 5.60(3/5H, s) 4.11(1H, |
| | | | dd, J=15.8, 7.9) 4.01(3H, s) 3.67(6/5H, |
| | | | q, J=6.8) 3.31(4/5H, q, J=6.9) 2.88- |
| | | | 2.66(4H, m) 2.54(6/5H, q, J=7.4) 2.46 |
| | | | (4/5H, q, J==7.4) 2.21-2.02(4H, m) |
| 14 | 6j | 3,3-di- | ¹H NMR(300MHz, CDCl₃) 7.43-7.289 |
| | | phenyl- | (15H, m) 6.97(3/5H, d, J=7.9) 6.96 |
| | | propyl | (2/3H, d, J=8.0) 6.84-6.75(12/5H, |
| | | | m) 6.15(3/5H, t, 15.7) 5.77-5.60(1H, |
| | | | m) 5.62(2/5H, s) 5.60(3/5H, s) 4.11(1H, |
| | | | dd, J=15.8, 7.9) 4.01(3H, s) 3.67 |
| | | | (6/5H, q, J=6.8) 3.31(4/5H, q, J=6.9) |
| | | | 2.88-2.66(4H, m) 2.54(6/5H, q, J=7.4) |
| | | | 2.46(4/5H, q, J=7.4) 2.21-2.02(4H, m) |
| 15 | 6k | 3-benzyl- | ¹H NMR(300MHz, CDCl₃) 7.32-7.14 |
| | | 4-phenyl- | (15H, m) 6.85(6/11H, d, J=8.0) 67.84 |
| | | butyl | (5/11H, d, J=7.9) 6.71-6.63(27/11H, m) |
| | | | 5.83(6/11H, t, J=5.5) 5.61-5.54(1H, m) |
| | | | 5.50(5/11H, s) 5.48(6/11H, s) 3.87 |
| | | | (18/11H, s) 3.86(15/11H, s) 3.56 |
| | | | (12/11H, t, J=6.1) 3.15(10/11H, |
| | | | t, J=6.2) 2.76-2.51(8H, m) |
| | | | 2.40-2.33(12/11H, m) 2.28- |
| | | | 2.21(10/11H, m) 2.05-1.80(4H, m) |
| 16 | 6l | 2-(4-tri- | ¹1 NMR(300Mhz, CDCl₃) 7.59(2H, |
| | | fluoro- | d, J=18.0) 7.36(2H, d, J=8.0) 7.32- |
| | | methyl- | 7.16(5H, m) 6.84(1H, d, J=8.0) 6.71 |
| | | phenyl)- | (1H, d, J=1.9) 6.67(1H, dd, J=8.0, 1.9) |
| | | ehtyl | 6.53(1/3H, t, J=4.7) 6.10(2/3H, t, J=5.6) |
| | | | 5.63-5.55(1H, m) 5.48(1H, s) 3.89(2H, |
| | | | s) 3.88(1H, s) 3.83(4/3H, q, J=6.7) |
| | | | 3.43(2/3H, q, J=6.7) 3.03(4/3H, t, |
| | | | J=7.0) 2.88(2/3H, t, j=7.1) |
| | | | 2.73-2.58(4H, m) 2.06-1.95(4H, m) |
| 17 | 6m | 2-pyridin- | ¹H NMR(300MHz, CDCl₃) 8.49-8.47 |
| | | 2-yl-e | (1H, m) 7.57-7.52(1H, m) 7.21-7.07 |
| | | | (8H, m) 6.75(1/3H, d, J=8.0) 6.74 |
| | | | (2/3H, d, J=8.0) 6.63-6.52(2H, |
| | | | m) 5.61-5.47(2H, m) 3.90(4/3H, q, |
| | | | J=6.1) 3.80(3H, s) 3.55(2/3H, q, |
| | | | J=6.3) 3.03(4/3H, t, J=6.3) 2.89(2/3H, |
| | | | t, J=6.5) 2.64-2.49(4H, m) 2.02- |
| | | | 1.75(4H, m) |
| 18 | 6n | 2-thiophen- | ¹H NMR(300Mhz, CDCl₃) 7.27-7.15 |
| | | 2-yl-ethyl | (6H, m) 6.97-6.93(1H, m) 6.86-6.81 |
| | | | (2H, m) 6.70-6.63(7/3H,m) 6.22 |
| | | | (2/3H, t, J=5.9) 5.64-5.57(1H, |
| | | | m) 5.46(1H, s) 3.88(3H, s) 3.84(4/3H, |
| | | | q, J=6.4) 3.46(2/3H,q, t, J=6.6) |
| | | | 3.17(4/3H, t, J=6.6) 3.03(2/3H, t, |
| | | | J=6.9) 2.72-2.57(4H, m) 2.10- |
| | | | 1.87(4H, m) |
| 19 | 6o | 2-penta- | ¹H NMR(300Mhz, CDCl₃) 7.16-7.02 |
| | | fluoro- | (5H, m) 6.69(1H, d, J=8.0) 6.59-6.51 |
| | | phenylethyl | (7/3H, m) 6.04(2/3H, t, J=6.0) 5.50- |
| | | | 5.34(1H, m) 5.34(1H, s) 3.75(3H, s) |
| | | | 3.69(4/3H, q, J=6.6) 3.24(2/3H, |
| | | | q, J=6.8) 2.93(4/3H, t, J=6.6) |
| | | | 2.81(2/3H, t, J=7.4) 2.56-2.43 |
| | | | (4H, m) 1.92-1.78(4H, m) |
| 20 | 6p | 2-(2-fluoro- | ¹H NMR(300MHz, CDCl₃) 7.19-6.96 |
| | | phenyl)ethyl | (9H, m) 6.75(1H, d, J=8.0) 6.64- |
| | | | 6.56(7/3H, m) 6.08(2/3H, t, J=5.7) |
| | | | 5.57-5.47(1H, m) 5.38(1H, s) 3.80 |
| | | | (3H, s) 3.74(4/3H, q, J=6.7) 3.38 |
| | | | (2/3H, q, J=6.7) 2.92(4/3H, t, J=6.8) |
| | | | 2.79(2/3H, t, J=7.0) 2.64-2.48(4H, m) |
| | | | 2.01-1.88(4H, m) |
| 21 | 6q | 2-(3-fluoro- | IR (neat) 3542, 3398, 3023, 2937, |
| | | phenylethyl | 1590; MS(CI) 468(M³⁰ + 1) |
| 22 | 6r | Naphthalen- | ¹NMR(300MHz, CDCl₃) 8.38(1H, s), |
| | | 2-yl | 7.74-7.68(4H, m), 7.43-7.23(3H, m), |
| | | | 7.20-7.08(5H, m), 6.73(1H, d, J=7.8), |
| | | | 6.58(2H, m), 5.69(1H, m), 5.39(1H, s), |
| | | | 3.73(3H, s), 2.67-2.57(4H, m), 2.09- |
| | | | 1.92(4H, m) |
| 23 | 6s | Naphthalen- | IR (neat) 3533, 3390, 3025, 2950, |
| | | 2-yl | 1602; Mass(CI) 486(M⁺+ 1) |
| 24 | 6t | 4-t- | IR (neat) 3532, 3356, 3026, 2950, |
| | | butyl- | 1604; Mass(CI) 506(M⁺+ 1) |
| | | phenethyl | |

### EXAMPLE 25

### Synthesis of 3-[4-(t-butyl-dimethyl-silanyloxy)-3-methoxyphenyl]-1-phenyl-prop-2-en-1-ol (7a) (R₂=Phenyl)

Compound 2(486 mg, 1.66 mmol) was diluted in tetrahydrofuran (9 ml), and the solution was added, through cannular, to the flask filled with argon gas, and then cooled to −78° C. Phenyl magnesium bromide (1.0M solution dissolved in IBF, 3.32 ml, 3.32 mmol) was slowly added thereto, and the mixed was stirred for 30 minutes and then cooled to room temperature, followed by stirring for 2 hours. After the completion of the reaction was confirmed using TLC, saturated aqueous NH₄Cl solution was added thereto to terminate the reaction. The reaction mixture was extracted with ethyl acetate (60 ml). The organic layer was washed successively with saturated aqueous NH₄Cl solution (7 ml), water (7 ml×3) and brine (7 ml), and then dried over anhydrous Na₂SO₄. The resulting mixture was concentrated under reduced pressure and the obtained residue was column-chromatographed (hexane/ethyl acetate=20/1) to yield 254 mg (41.2%) of the compound as a colorless oil.

R_{f}=0.27 (n-hexane: EtOAc=5:1, SiO₂) UV/anisaldehyde: IR (neat) 3368, 3030, 2955, 2930, 2857, 1651, 1601, 1577, 1512, 1416 cm⁻¹

### EXAMPLE 26

### Synthesis of 3-[4(t-butyl-dimethyl-silanyloxy)-3-methoxy-phenyl]-1-phenyl-propan-1-ol (8a) (R₂=Phenyl)

Compound 7a (232 mg, 0.63 mmol) was dissolved in ethanol, and palladium/carbon (40 mg) was added thereto, followed by stirring at hydrogen gas atmosphere. After 2 hours, the completion of the reaction was confirmed. The resulting mixture was filtered to remove the Pd/C, and concentrated under reduced pressure. The obtained residue was column-chromatographed (hexane/ethyl acetate=20/1) to yield the pure compound (140 mg, 60.1%) as a colorless oil.

IR (neat) 3359, 3029, 2857, 1601, 1577, 1512, 1464, 1281 cm⁻¹; ¹H NMR (300 MHz, CDCl₃) 7.22-7.12(5H, m), 6.61(1H, d, J=7.9), 6.53(1H, d, J=1.9), 6.49(1H, dd, J=7.9, 1.9), 4.54(1H, dd, J=7.8, 5.3), 3.64(3H, s),-2.60-2.44(2H, m), 1.99-1.87(2H, m), 1.42(1H, s), 1.85(9H, s), 0.00(611, s).

### EXAMPLE 27

### Synthesis of Benzyl-Carbamate 3-[4-(t-butyl-dimethyl-silanyloxy)-3-methoxy-phenyl]-1-phenyl-propyl Ester (9a) (R₁=Benzyl, R₂=Phenyl)

A solution of compound 8a (140 mg, 0.38 mmol) in toluene (7 ml) was added to the flask filled with nitrogen gas, and benzyl isocyanate (0.13 ml, 1.05 mmol) was added thereto, followed by heating at reflux. After 24 hours, the completion of the reaction was confirmed. The reaction mixture was concentrated under reduced pressure to remove the toluene, diluted with ethyl acetate (50 ml), washed successively with 5% aqueous ammonia (6 ml×2), saturated saline solution (6 ml×4), and then dried over anhydrous sodium sulfate. The resulting mixture was concentrated under reduced pressure and the obtained residue was column-chromatographed (hexane/ethyl acetate=20/1) to yield the compound (139 mg, 73.1%) as a colorless oil.

IR (neat) 3342, 3033, 2953, 2930, 2857, 1714, 1605, 1585, 1514, 1454, 1253 cm⁻¹; ¹H NMR (300 MHz, CDCl₃) 7.22-7.12(10H, m), 6.60(1H, d, 3=7.9), 6.48-6.44(211), 5.58(1H, dd, J=7.7, 5.9), 4.92-4.90(1H, m), 4.29-4.14(21, m), 3.63(3H, s), 2.53-2.35(2H, m), 2.15-2.03(1H, m), 1.97-1.85(1H, m), 0.85(9H,s), 0.00(6H, s)

### EXAMPLE 28

### Synthesis of Benzyl-Carbamate 3-(4-hydroxy-3-methoxy-phenyl)-1-phenyl-propyl Ester (10a) (R₁=Benzyl, R₂=Phenyl)

Compound 9a (132 mg, 0.26 mmol) was dissolved in THF (6 ml), and tetrabutylammonium fluoride (1M solution dissolved in THF, 0.65 ml, 0.65 mmol) was slowly added thereto, followed by sting for 20 minutes and then confirming the completion of the reaction using TLC. The resulting mixture was extracted with ethyl acetate. The obtained organic layer was washed successively with water (4 ml×2) and brine (4 ml), dried over Na₂SO₄, and then concentrated under reduced pressure. The obtained residue was column-chromatographed (hexane/ethyl acetate=4/1) to yield the pure compound (72 mg, 70.6%) as a colorless oil.

IR (neat) 3531, 3354, 3036, 2938, 1700, 1604, 1516, 1455, 1266 cm⁻¹; ¹H NMR (300 MHz, CDCl₃) 7.26-7.17(10H, m). 6.74(1H, d, J=8.5), 6.57-6.55(2H), 5.64(11H, dd, J=7.8, 5.9), 5.40(1H, s), 4.98(1H, m), 4.35-4.20(2 h, m), 3.77(3 h, s), 2.54-2.44(2 h, m), 2.16-2.11(1H, m), 2.00-1.90(1H, m); MS(EI) m/e(relative intensity) 391(M⁺) 240(100) 137(39) 91(23)

Compounds 10b˜f were synthesized according to the similar procedure as the Example 28, and parts of spectral data thereof are shown below.

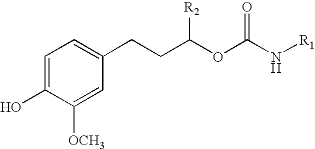

| Examples | Compounds | R₁ | R₂ | Spectral data |
|---|---|---|---|---|
| 29 | 10b | Benzyl | phenethyl | ¹H NMR(300MHz, CDCl₃) 7.37-7.15(10H, m), |
| | | | | 6.84(1H, d, J=8.0), 6.73-6.59(2H, m), 6.35(1H, t, |
| | | | | J=5.33), 5.68-5.60(1H, m), 5.49(1H, s), 4.77(1H, d, |
| | | | | J=5.7), 4.36(1H, d, J=5.7), 3.89(3H, s), |
| | | | | 2.70-2.56(4H, m), 2.07-1.94(4H, m) |
| 30 | 10c | benzyl | ethyl | ¹H NMR (300MHz, CDCl₃) 7.37-7.26(5H, m), |
| | | | | 6.82(1H, d, J=8.2), 6.70-6.63(2H, m), 5.47(1H, s), |
| | | | | 4.96(1H, b), 4.80(1H, quint, J=6.1), 4.38(2H, d, |
| | | | | J=5.8), 3.86(3H, s), 2.63-2.50(2H, m), 1.89-1.77(2H, |
| | | | | m), 1.66-1.56(2H, m), 0.94-0.87(3H, m) |
| 31 | 10d | benzyl | methyl | ¹H NMR (300MHz, CDCl₃) 7.30-7.18(5H, m), |
| | | | | 6.75(1H, d, J=8.6), 6.60-6.58(2H), 5.41(1H, s), |
| | | | | 4.90-4.76(2H, m), 4.32-4.30(2H, m), 3.79(3H, s), |
| | | | | 2.53-2.48(2h, m), 1.83-1.67(2H, m) 1.19(3H, d, J= |
| | | | | 6.20) |
| 32 | 10e | penethyl | phenethyl | ¹H NMR(300MHz, CDCl₃) 7.37-7.16(10H, m), |
| | | | | 6.84 (1H, d, J=8.8), 6.69 (1H, d, J=1.8), 6.67 (1H, |
| | | | | dd, J=8.8, 1.8), 5.50 (1H, s), 4.93-4.87 (1H, m), 4.66 |
| | | | | (1H, m), 3.88(1H, s), 3.48 (2H, dd, J=13.2, 6.6), 2.85 |
| | | | | (2H, t, J=6.9), 2.70-2.54 (4H, m), 1.90-1.82 (4H, m) |
| 33 | 10f | phenethyl | ethyl | ¹H NMR (300MHz, CDCl₃) 7.26-7.11(5H, m), |
| | | | | 6.75(H, d, J=8.0), 6.60-6.57(2H, m), 5.44(1H, s), |
| | | | | 4.69-4.64(1H, m), 4.58(1H, b), 3.79(3H, s), 3.37(2H, |
| | | | | q, J=6.4), 2.74(2H, J=6.9), 2.53-2.43(2H, m), |
| | | | | 1.72-1.70(2H, m), 1.52-1.47(2H, m), 0.84-0.76(3H, |
| | | | | m) |

### EXAMPLE 34

### Synethesis of {3-[4-(t-butyl-dimethyl-silanyloxy)-3-methoxphenyl]-1-phenethyl-propyl}-methyl amine (12a) (R₂=Phenethyl, R₃=Methyl)

Methylamine (0.13 ml, 0.065 mmol, 2M solution in methanol) was poured into a flask, and then methylamine.HCl (8.84 mg, 0.13 mmol) and NaBH₃CN were successively added thereto, followed by stirring. A solution of compound 11 (52.2 mg, 0.13 mmol) in methanol was slowly added thereto and stirred for 5 days. After confirming disappearance of compound 11 using TLC, the reaction mixture was concentrated under reduced pressure to remove the methanol, and then extracted with ethyl acetate. The organic layer was washed successively with water and saturated aqueous sodium chloride solution, dried over anhydrous Na₂SO₄, and then concentrated under reduced pressure to yield the impure compound 12a (52.3 mg) as a colorless oil.

IR(n eat) 3300 cm⁻¹; MS(CI) 414(M⁺+1)

### EXAMPLE 35

### Synthesis of 1-3-[4(t-butyl-dimethyl-silanyloxy)-3-methoxy-phenyl]-1-phenethyl-propyl-1-meth yl-3-phenethyl-thiourea (13a) (R₂=Phenethyl, R₃=Methyl)

Compound 12a (52.3 mg, 0.13 mmol) was diluted in toluene (6 ml), and the solution was poured into a flask. Phenethyl isothiocyanate (57 μl, 0.38 mmol) was added thereto, followed by heating at reflux for 48 hours. The reaction mixture was concentrated under reduced pressure to remove the toluene, diluted with ethyl acetate (40 ml), washed successively with 5% aqueous ammonia (6 ml×2) and saturated aqueous sodium chloride solution (6 ml×4), and then dried over Na₂SO₄. The resulting mixture was concentrated under reduced pressure and the obtained residue was column-chromatographed (n-hexane/ethyl acetate=12/1, SiO₂) to yield the compound (45.5 mg, 62.4%) as a colorless oil.

IR (neat) 3420, 3026, 2931, 1604, 1514, 1386, 1282 cm⁻¹; ¹H NMR (300 MHz, CDCl₃) 7.16-6.98(10H, m) 6.59(1H, d, J=8.0) 6.52(1H, d, J=1.8) 6.42(1H, dd, J=8.0, 1.8) 4.94(1H, s) 3.77-3.75(2H, m) 3.64(3H, s) 2.77(2H, m) 2.50(3H, s) 2.40-2.22(4H, m) 1.71-1.59(4H, m)

### EXAMPLE 36

### Synthesis of 1-[3-(hydroxy-3-methoxy-phenyl)-1-phenethyl-propyl]-1-methyl-3-phenethyl-thiou rea (14a) (R₂=Phenethyl, R₃=Methyl)

Compound 13a (37 mg, 0.06 mmol) was dissolved in THF (6ml), and tetrabutylammonium fluoride (1M-THF solution, 0.16 ml, 0.16 mmol) was slowly added thereto, followed by stirring for 20 minutes and then confirming the completion of the reaction using TLC. The reaction mixture was extracted with ethyl acetate. The obtained organic layer was washed successively with water (4 ml×2) and saturated aqueous sodium chloride solution (4 ml), dried over Na₂SO₄, and then concentrated under reduced pressure. The obtained residue was column-chromatographed (n-hexane/ethyl acetate=4/1, SiO₂) to yield the pure compound 14a (14 mg, 47.6%) as a colorless oil.

IR (neat) 3538, 3417, 3024, 2936, 1604, 1517, 1453, 1331, 1270 cm⁻¹; ¹H NMR (300 MHz, CDCl₃) 7.17-6.98(10H, m) 6.68(1H, d, J=8.0) 6.57(1H, d, J=1.7) 6.17(1H, dd, J=8.0, 1.7) 5.34(1H, s) 4.94(1H, s) 3.74(5H, m) 2.76(2H, m) 2.54(3H, s) 2.41-2.28(4H, m) 1.77-1.54(4H, m)

Compounds 14b˜d were synthesized according to the similar procedure as the Example 36, and parts of spectral data thereof are shown below.

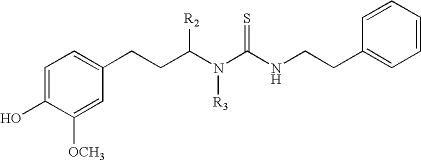

| Examples | Compounds | R₂ | R₃ | Spectral data |
|---|---|---|---|---|
| 37 | 14b | Phenethyl | H | IR(neat) 3531, 3265, 3026, 2940, 1604, 1546, 1515, |
| | | | | 1453, 1270, 1234 cm⁻¹; MS (CI) 449(M⁺+ H) 432(19) |
| | | | | 328(100) 286(91) 269(85) 105(74) |
| 38 | 14c | Ethyl | Phenethyl | IR(neat) 3530; MS(CI) 477(M⁺+ H) |
| 39 | 14d | Ethyl | Benzyl | ¹H NMR(300Mhz, CDCl₃) 7.34-6.98(10H, m), |
| | | | | 6.81(1H, d, J=8.0), 6.65(1H, d, J=1.9), 6.59(1H, dd, |
| | | | | J=8.0, 1.9), 5.45(1H, b), 5.32(1H, m), 4.48(2H, q, |
| | | | | J=15.5), 3.92-3.87(2H, m), 3.86(3H, s), 2.75(2H, t, |
| | | | | J=6.8), 2.68-2.54(2H, m), 1.75(2H, q, J=7.6), |
| | | | | 1.61-1.51(2, m), 0.92(3H, t, J=7.3) |

### EXAMPLE 40

### Synthesis of Phenethyl-Thiocarbamic Acid O-3-[4(t-butyl-dimethyl-silanyloxy)-3-methoxy-phenyl]-1-ethyl-propyl Ester (15c) R₂=Ethyl)

1-[4-(t-butyl-dimethyl-silanyloxy)-3-methoxy-phenyl]-3-pentan-3-ol (8c) (357.3 mg, 1.10 mmol) was dissolved in THF, and 60% NaH (154 mg, 3.5 equivalents) was added thereto, followed by stirring at 60° C. for 1 hour. A solution of phenethyl isothiocyanate (0.54 ml, 1 equivalent) in THF was added thereto, and the mixture was stirred at room temperature. After confirming the completion of the reaction using TLC, saturated aqueous ammonium chloride solution was added thereto to terminate the reaction. The reaction mixture was diluted in ethyl acetate, washed with water and saturated aqueous sodium chloride solution, dried over anhydrous sodium sulfate, and then concentrated under reduced pressure. The obtained residue was column-chromatographed (n-hexane/ethyl acetate=30/1) to yield the compound (430.9 mg, 80.3%) as a pale yellow liquid.

IR (neat) 3359, 3033, 2931, 2857, 1514, 1464, 1279 cm⁻¹; ¹H NMR (300 MHz, CDCl₃) 7.21-7.03(m, 51) 6.61(d, 2/5H, J=8.0), 6.60(d, 3/5H, J=8.0), 6.55-6.54(m, 1), 6.50-6.45(m, 1H), 6.43(t, 2/5H, J=5.1), 6.01(t, 3/5H, J=5.4), 3.65(s, 9/5H), 3.64(s, 6/5H), 3.71-3.63(m, 6/5H), 3.41-3.32(m, 4/5H), 2.80(t, 6/5H, J=7.0), 2.68(t, 4/5H, J=7.2), 2.51-2.38(m, 2H), 1.92-1.70(m, 2H), 1.641.47(m, 2H), 0.85(s, 9H), 0.83-0.73(m, 3H), 0.00(s, 6H).

### EXAMPLE 41

### Synthesis of Phenethyl-Thiocarbamic Acid O-[1-ethyl-3-(4-hydroxy-3-methoxy-phenyl)-propyl]ester (16c) (R₂=Ethyl)

Phenethyl-Thiocarbamic acid O-3-[4-(t-butyl-dimethyl-silanyloxy)-3-methoxy-phenyl]-1-ethyl-propylester (15c) (415 mg, 0.85 mmol) was dissolved in THF (10 ml), and to the solution was slowly added tetrabutylammonium fluoride (1M-solution dissolved in THF, 1.5 ml, 1.5 mmol), followed by stirring for 15 minutes and confining the compleltion of the reaction using the TLC. The reaction mixture was extracted successively with ethyl acetate. The obtained organic layer was washed successively with water (4 ml×2) and saturated aqueous sodium chloride solution (4 ml), dried over anhydrous Na₂SO₄, and then concentrated under reduced pressure. The obtained residue was column-chromatographed (n-hexane/ethyl acetate=7/1) to yield the pure compound (292.4 mg, 91.9%) as a colorless oil.

IR (neat) 3522, 3364, 2937, 1604, 1514, 1454, 1270, 1231 cm⁻¹; ¹H NMR (300 MHz, CDCl₃) 7.34-7.21(m, 5H:) 6.85(d, 2/5H, J=8.0) 6.84(d, 3/5H, J=8.0) 6.6.75-6.67(m, 2H) 6.60(m, 2/5H) 6.17(m, 3/5H) 5.55-5.40(m, 2H) 3.90(s, 9/5H) 3.89(s, 6/5H) 3.88-3.81(m, 6/5H) 3.543.⁴6(m, 4/5H) 2.96(t, 6/5H, J=7.0) 2.84(t, 4/5H, J=7.2) 2.67-2.57(m, 2H) 2.07-1.87(m, 2H) 1.78-1.64(m, 2H) 0.99-0.90(m, 3H).

Compounds 16a˜b and 16d˜p were synthesized according to the similar procedure as Example 41, and parts of spectral data thereof are shown below.

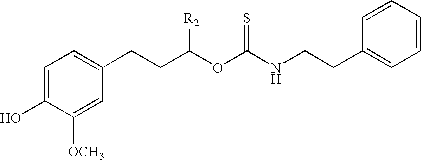

| Examples | Compounds | R₂ | Spectral data |
|---|---|---|---|
| 42 | 16a | H | ¹H NMR(300MHz, CDCl₃) 7.25-7.07 |
| | | | 6.76-6.71(m, 1H) 6.59-6.55(m, 2H) 6.53(s, 3/8H) |
| | | | 6.20(s, 5/8H) 5.38(s, 1H), 4.40(t, 6/8H, J=6.5) 4.35(t, |
| | | | 10/8H, J=6.5) 3.77(s, 3H), 3.75-3.68(m, 10/8H) |
| | | | 3.44-3.37(m, 6/8H), 2.83(t, 10/8H, J=7.0) 2.72(t, 6/8H, |
| | | | J=7.1), 2.58-2.49(m, 2H) 1.96-1.83(m, 2H) |
| 43 | 16b | metyl | ¹H NMR(300MHz, CDCl₃) 7.25-7.11(m, 5H) 6.76 |
| | | | (d, 3/8H, J=7.9) 6.75(d, 5/8H, J=7.9), 6.63-6.57(m, |
| | | | 2H) 6.45(m, 3/8H) 6.04(m, 5/8H) 5.50-5.41(m, |
| | | | 1H), 5.37(s, 1H) 3.81(s, 10/8H) 3.80(s, 6/8H) |
| | | | 3.78-3.70(m, 10/8H), 3.44-3.36(m, 6/8H) 2.86(t, |
| | | | 10/8H, J=7.0) 2.74(t, 6/8H, J=7.2), 2.58-2.48(m, 2H) |
| | | | 2.01-1.68(m, 2H) 1.26(d, 9/8H, J=6.3), 1.21(d, |
| | | | 15/8H, J=6.3). |
| 44 | 16d | propyl | ¹H NMR(300MHz, CDCl₃) 7.20-7.02(m, 5H) |
| | | | 6.70-6.67(m, 1H) 6.58-6.50(m, 2H) 6.41(m, 2/5H) |
| | | | 5.99(m, 3/5H) 5.43-5.38(m, 1H) 5.32(s, 1H) 3.74(s, |
| | | | 3H), 3.74-3.67(m, 6/5H) 3.39-3.26(m, 4/5H), 2.80(t, |
| | | | 6/5H, J=6.8) 2.70(t, 4/5H, J=7.0) 2.47-2.41(m, 2H) |
| | | | 1.78-1.66(m, 2H), 1.59-1.39(m, 2H) 1.29-1.11(m, 2H) |
| | | | 0.82-0.75(m, 3H) |
| 45 | 16e | iso-propyl | ¹H NMR(300MHz, CDCl₃) 7.22-7.10(m, 5H) 6.72(d, |
| | | | 1H, J=8.0), 6.63-6.55(m, 2H) 6.47(m, 2/5H) 6.06(m, |
| | | | 3/5H), 5.39-5.29(m, 2H), 3.78(s, 9/5H), |
| | | | 377(s, 6/5H), 3.77-3.66(m, 6/5H) 3.43-3.35(m, 4/5H) |
| | | | 2.85(t, 6/5H, J=6.9), 2.73(t, 4/5H, J=7.1), 2.54-2.45(m, |
| | | | 2H), 1.97-1.72(m, 3H) 0.87-0.79(m, 6H). |
| 46 | 16f | iso-butyl | ¹ NMR(300MHz, CDCl₃) 7.22-7.10(m, 5H) 6.72(d, |
| | | | 1H, J=8.0), 6.63-6.55(m, 2H) 6.47(m, 2/5H) 6.06(m, |
| | | | 3/5H), 5.39-5.29(m, 2H) 3.78(s, 9/5H) 3.77(s, 6/5H), |
| | | | 3.77-3.66(m, 6/5H) 3.43-3.35(m, 4/5H) 2.85(t, 6/5H, |
| | | | J=6.9), 2.73(t, 4/5H, J=7.1) 2.54-2.45(m, 2H) |
| | | | 1.97-1.72(m, 3H) 0.87-0.79(m, 6H). |
| 47 | 16g | cyclo-propyl | ¹NMR(300MHz, CDCl₃) 7.20-7.03(m, 5H) 6.68(dd, |
| | | | 1H, J=8.1, 2.7), 6.62(d, 1H, J=1.8), .55(d, 1H, J=8.1), |
| | | | 6.34(m, 2/5H) 5.98(m, 3/5H), 3.75(s, 9/5H) 3.73(s, |
| | | | 4/5H) 3.61(q, 6/5H, J=6.6) 3.31(q, 4/5H, J=6.8), 2.78(t, |
| | | | 6/5H, J=7.0) 2.67(t, 4/5H, J=7.2) 2.49-2.42(m, 2H), |
| | | | 2.26-2.18(m, 2H) 1.54(d, J=13.9, 6H). |
| 48 | 16h | cyclo-butyl | ¹H NMR(300MHz, CDCl₃) 7.20-7.02(m, 5H) |
| | | | 6.70-6.67(m, 1H) 6.58-6.50(m, 2H), 6.41(m, 2/5H) |
| | | | 5.99(m, 3/5H) 5.43-5.38(m, 1H) 5.32(s, 1H), 3.74(s, |
| | | | 3H), 3.74-3.67(m, 6/5H) 3.39-3.26(m, 4/5H), 2.80(t, |
| | | | 6/5H, J=6.8) 2.70(t, 4/5H, J=7.0) 2.47-2.41(m, 2H) |
| | | | 1.78-1.66(m, 2H), 1.59-1.39(m, 2H) 1.29-1.11(m, 2H) |
| | | | 0.82-0.75(m, 3H). |
| 49 | 16i | hexyl | ¹1 NMR(300MHz, CDCl₃) 7.24-7.09(m, 5H) 6.75(d, |
| | | | 2/5H, J=8.0) 6.75(d, 3/5H, J=8.0), 6.65(d, 2/5H, J=1.9) |
| | | | 6.63(d, 3/5H, J=1.9) 6.61(dd, 2/5H, J=1.9, 8.0), 6.59(dd, |
| | | | 3/5H, J=1.9, 8.0) 6.46(t, 2/5H, J=5.7) 6.04(t, 3/5H, |
| | | | J=5.7), 5.50-5.40(m, 1H) 5.37(s, 1H) 3.80(s, 9/5H) |
| | | | 3.79(s, 6/5H) 3.78-3.71(m, 6/5H) 3.44-3.34(m, 4/5H) |
| | | | 2.86(t, 6/5H, J=7.0), 2.74(t, 4/5H, J=7.1) 2.62-2.45(m, |
| | | | 2H) 1.95-1.70(m, 2H) 1.70-1.45(m, 2H), 1.24-1.21(m, |
| | | | 6H) 0.83-0.78(m, 3H). |
| 50 | 16j | phenyl | ¹NMR(300MHz, CDCl₃) 7.40-7.18(m, 10H) 6.85(d, |
| | | | 1/3H, J=8.0) 6.84(d, 2/3H, J=8.0) 6.71-6.65(m, 2H) |
| | | | 6.65-6.40(m, 1/3H) 6.48-6.42(m, 1H), 6.30(t, 2/3H, |
| | | | J=5.3) 5.45(s, 1H) 3.89(s, 6/3H) 3.88(s, 3/3H), 3.81(q, |
| | | | 4/3H, J=6.6) 3.81(q, 2/3H, J=6.8) 2.94(t, 4/3H, J=7.0), |
| | | | 2.94(t, 2/3H, J=7.2) 2.69-2.57(m, 2H) 2.40-2.07(m, 2H) |
| 51 | 16k | benzyl | IR (NaCl/neat) 3517, 3363, 3027, 2936, 1604, 1515 |
| 52 | 16l | 3-phenylpropyl | ¹H NMR(300MHz, CDCl₃) 7.23-7.07(m, 10H) 6.74 |
| | | | (dd, 1H, J=1.4, 8.0) 6.63-6.46(m, 2H) 6.48(t, 1/3H, |
| | | | J=5.6) 6.04(t, 2/3H, J=5.6), 5.52-5.48(m, 1H) 5.37(s, |
| | | | 1H) 3.79(d, 3H, J=3.8) 3.76-3.68(m, 4/3H), 3.42-3.35 |
| | | | (m, 2/3H) 2.86(t, 4/3H, J=7.1) 2.72(t, 2/3H, J=7.1), |
| | | | 2.61-2.43(m, 4H) 1.93-1.74(m, 2H) 1.68-1.53(m, 4H) |
| 53 | 16m | 4-phenylbutyl | ¹H NMR(300MHz, CDCl₃) 7.24-7.07(m, 10H), |
| | | | 6.75(d, 2/5H, J=8.0), 6.75(d, 3/5H, J=8.0), 6.64-6.56(m, |
| | | | 2H) 6.45(t, 2/5H, J=5.8), 6.03(t, 3/5H, J=5.8), |
| | | | 5.49-5.40(m, 1H), 5.37(s, 1H), 3.80(s, 9/5H), 3.79(s, |
| | | | 6/5H), 3.78-3.71(m, 6/5H), 3.40-3.33(m, 4/5H), 2.86(t, |
| | | | 6/5H, J=6.9) 2.71(t, 4/5H, J=7.2) 2.56-2.46(m, 4H), |
| | | | 1.91-1.74(m, 2H), 1.68-1.49(m, 4H) 1.34-1.27(m, 2H) |
| 54 | 16n | 2-(4-trifluorome | ¹1 NMR(300Mhz, CDCl₃) 7.44(2H, d, J=8.0), |
| | | thylphenyl)ethyl | 7.28-7.09(7H, m), 6.76(1/3H, d, J=8.0), 6.75(2/3H, d, |
| | | | J=8.0), 6.63-6.56(2H, m), 6.49(1/3H, t, J=5.8), |
| | | | 6.05(2/3H, t, J=5.8), 5.55-5.48(1H, m), 5.41(1/3H, s), |
| | | | 5.40(2/3H, s), 3.79(6/3H, s), 3.78(3/3H, s), 3.74(4/3H, q, |
| | | | J=7.1), 3.36(2/3H, q, J=6.7), 2.87(4/3H, t, J=7.0), |
| | | | 2.73(2/3H, t, J=7.2), 2.68-2.48(4H, m), 2.00-1.76(4H, m) |
| 55 | 16o | 2-(4-bromophen | ¹H NMR(300MHz, CDCl₃) 7.37-7.17(9H, m), |
| | | | 6.86(1/3H, d, J=8.0), 6.85(2/3H, d, J=8.0), 6.73-6.67(2H, |
| | | | m), 6.62(1/3H, t, J=5.6), 6.15(2/3H, t, J=5.6), |
| | | | 5.66-5.60(1H, m), 5.51(1/3H, s), 5.50(2/3H, s), |
| | | | 3.90(6/3H, s), 3.89(3/3H, s), 3.84(4/3H, q, J=6.7), |
| | | | 3.46(2/3H, q, J=6.8), 2.96(4/3H, t, J=7.0), 2.84(2/3H, t, |
| | | | J=7.2), 2.74-2.58(4H, m), 2.13-1.89(4H, m) |
| 56 | 16p | dimethyl | ¹H NMR(300MHz, CDCl₃) 7.20-7.03(m, 5H), |
| | | | 6.68(dd, 1H, 21=8.1, 2.7), 6.62(d, 1H, J=1.8) 6.55(d, 1H, |
| | | | J=8.1), 6.34(m, 2/5H) 5.98(m, 3/5H), 3.75(s, 9/5H), |
| | | | 3.73(s, 4/5H) 3.61(q, 6/5H, J=6.6), 3.31(q, 4/5H, J=6.8), |
| | | | 2.78(t, 6/5H, J=7.0), 2.67(t, 4/5H, J=7.2), 2.49-2.42(m, |
| | | | 2H), 2.26-2.18(m, 2H), 1.54(d, 21=13.9, 6H). |

### EXAMPLE 57

### Synthesis of 1-[4-(t-butyl-dimethyl-silanyloxy)-3-methoxy-phenyl]-ethanone (19)

Acetovanillone (18) (1.0306 g, 6.202 mmol) was dissolved in THF, and the solution was poured into the dry flask filled with argon gas. To the mixture was add imidazole (1.056 g, 15.505 mmol), and a solution of TBSCl (2.337 g, 15.505 mmol) in THF was slowly added thereto, followed by stirring at 60° C. for 16 hours. After the completion of the reaction, the reaction mixture was extracted with ethyl acetate. The obtained organic layer was washed with H₂O and brine, dried over Na₂SO₄, and then evaporated under reduced pressure. The obtained residue was column-chromatographed (hexane/ethyl acetate=6/1) to yield the compound 19 (1.38 g, 79.2%) as a pale yellow solid.

IR (neat) 3013, 2956, 2931, 2858, 1676, 1593, 1509, 1287 cm⁻¹; ¹H NMR (300 MHz, CDCl₃)δ 7.33(1H, d, J=2.0), 7.31(1H, d.d, J=2.0, 8.1), 6.70(1H, d, J=8.1), 3.68(3H, s), 2.38(3H, s), 0.82(9H, s), 0.00(6H, s)

### EXAMPLE 58

### Synthesis of Acetic Acid 4-(t-butyl-dimethyl-silanoxy)-3-methoxy-phenyl Ester (20)

1-[4(t-butyl-dimethyl-silanyloxy)-3-methoxy-phenyl]-ethanone (19) (567.0 mg, 2.02 mmol) was dissolved in methylene chloride. The solution was poured into the dry flask filled with argon gas, and m-CPBA (1003.7 mg, 6.06 mmol) and sodium bicarbonate (488.9 mg, 6.06 mmol) were added thereto in sequence, followed by heating at reflux with stirring. After 3 hours, 10% aqueous sodium bisulfate solution was added to a white reaction mixture in the form of suspension to decompose the excess m-CPBA, and an orgnic layer was partitioned and collected with melhylene chloride. The collected organic layer was washed successively with saturated aqueous potassium carbonate solution and saturated aqueous sodium chloride solution, and evaporated under reduced pressure. The obtained residue was column-chromatographed (hexane/ethyl acetate—15/1). (yield: 79.4%)

IR(NaCl/neat) 3503, 2955, 2931, 2886, 2857, 1766, 1508, 1209 cm⁻¹; ¹H N (300 MHz, CDCl₃) 6.66(d, 1H, J=8.5), 6.46(d, 1H, J=2.6), 6.41(d.d, 1K, J=2.6, 8.5), 3.63(s, 3H), 2.12(s, 3H), 0.84(s, 9H), 0.00(s, 6H)

### EXAMPLE 59

### Synthesis of 4(t-butyl-dimethyl-silanyloxy)-3-methoxy-phenol (21)

Acetic acid 4-(t-butyl-dimethyl-silanoxy)-3-methoxy-phenyl ester (20) (218.8 mg, 0.74 mmol) was poured into a flask and dissolved in methanol. To the solution was added potassium bicarbonate (147.8 mg, 1.48 mmol), followed by stirring. After confirming the completion of the reaction, the reaction mixture was evaporated under reduced pressure to remove the methanol, extracted with ethyl acetate. The obtained organic layer was washed with water and brine, and then evaporated under reduced pressure. The obtained residue was column-chromatographed (hexane/ethyl acetate=15/1). (yield: 94.2%)

IR(NaCl/neat): 3391, 2955, 2931, 2894, 2858, 1601, 1510, 1230 cm⁻¹; ¹H NMR (300 MHz, CDCl₃) 6;57(d, 1H, J=8.5 Hz), 6.29(d, 1H, J=2.8 Hz), 6.13(dd, 1H, J=2.8 Hz, 8.5 Hz), 3.64(s, 3H), 0.86(s, 9H), 0.00(s, 61).

### EXAMPLE 60

### Synthesis of t-butyl-(2-methoxy-4-oxiranylmethoxy-phenoxy)-dimethyl-silane (22)

60% NaH in oil (56.3 mg, 1.407 mmol) was poured into a dried flask and the flask was filled with argon gas. Then, a solution of 4-(t-butyl-dimethyl-silanyloxy)-3-methoxy-phenol (21) (102.3 mg, 0.402 mmol) in THF was poured into the flask. After heating at reflux for 1 hour with stirring, the mixture was cooled to room temperature and epichlorohydrin (111.6 mg, 0.94 ml, 1.206 mmol) was added thereto, followed by heating at reflux for 16 hours with string. The reaction was quenched with saturated aqueous ammonium chloride solution, and the reaction mixture was extracted with ethyl acetate. The obtained organic layer was washed with water and brine, and then evaporated under reduced pressure. The obtained residue was column-chromatographed (hexane/ethyl acetate=15/1). (yield 44.7%)

IR (neat) 3017, 2957, 2930, 2858, 1592, 1508, 1472, 1271, 1228 cm⁻¹

### EXAMPLE 61

### Synthesis of 1-[4-(t-butyl-dimethyl-silanyloxy)-3-methoxy-phenoxy]-propan-2-ol (23)

t-Butyl-(2-methoxy-4-oxiranylmethoxy-phenoxy)-dimethyl-silane (21) (19.6 mg, 0.063 mmol) was poured into a flask and dissolved by addition of methanol, and to the solution was added Pd-C (3 mg), followed by sting at hydrogen gas atmosphere. The reaction mixture was filtered through cellite under reduced pressure and then evaporated under reduced pressure. The obtained residue was column-chromatographed (hexane/ethyl acetate=6/1). (yield: 92.4%)

IR (NaCl, neat) 3420, 2955, 2930, 2879, 2858, 1591, 1510, 1450, 1270, 1228 cm⁻¹; ¹H NMR (300 MHz, CDCl₃) δ 6.71(1H, d, J=8.8), 6.45(1H, d, J=2.8), 6.30(1H, d.d, J=2.8, 8.8), 3.91-3.87(2H, m), 3.79-3.73(1H, m), 3.75(3H, s), 2.36(1H, s, —OH), 1.00(3H, t, J=7.2), 0.96(9H, s), 0.10(6H, s).

### EXAMPLE 62

### Synthesis of Phenethyl-Thiocarbamate O-2-[4-(t-butyl-dimethyl-silanyloxy)-3-methoxy-phenoxy]-1-methyl-ethylester (24)

60% NaH in oil (8.2 mg, 0.203 mmol) was poured into a dried flask and the flask was filled with argon gas. Then, a solution of 1-[4-(t-butyl-dimethyl-silanyloxy)-3-methoxy-phenoxy]-propan-2-ol (23) (18.2 mg, 0.058 mmol) in THF was poured into the flask, followed by stirring at 60° C. for 1 hour. At room temperature, phenethylisothiocyanate (8.5 mg, 26 μl, 0.174 mmol) was added thereto and stirred for 16 hours. After the completion of the reaction, the reaction mixture was extracted with ethyl acetate. The obtained organic layer was washed water and brine, and then evaporated under reduced pressure. The obtained residue was column-chromatographed (hexane/ethyl acetate=15/1). (yield: 81.2%)

IR(neat) 3358, 3027, 2931, 2857, 1510, 1450, 1227 cm⁻¹

### EXAMPLE 63

### Synthesis of Phenethyl-Thiocarbamate O-[2-(4-hydrxoy-2-methoxy-phenoxy)-1-methyl-ethyl] Ester (25) R₂=—CH₃)

Phenethylthiocarbamate O-2-[4-(t-butyl-dimethyl-silanyloxy)-3-methoxy-phenoxy]-1-methyl-ethyl ester (24) (14.4 mg, 0.030 mmol) was poured into a dried flask and dissolved by addition of THF. To the solution was slowly added 1M tetrabutylammonium fluoride (60 μl, 0.060 mmol), followed by stirring for 15 minutes. After the completion of the reaction, the reaction mixture was extracted with ethyl acetate. The organic layer was washed with water and brine, and evaporated under reduced pressure. The residue was column-chromatographed hexane/ethyl acetate=6/1). (yield: 75.9%)

IR(neat): 3537, 3387, 3025, 2937, 1611, 1511, 1228 cm⁻¹; ¹H NMR (300 MHz, CDCl₃): 7.35-7.15(m, 5H), 6.83(d, 1H, J=8.7), 6.56(m, 1H), 6.44-6.29(m, 1H), 4.104.03(m, 21), 3.88-3.80(m, 4H), 3.55-3.45(m, 1H), 2.95(t, 1.4H, J=7.0), 2.82(t, 0.6H, J=7.2), 1.59(s, 3H).

### EXAMPLE 64

### Synthesis of 1-(4-nitro-phenoxy)-butan-2-ol (27a) (R₄=NO₂, R₅=H)

4-Nitrophenol (1.8103 g, 13.01 mmol) was dissolved in 1,2-epoxybutane, and the solution was poured into a dried flask filled with argon gas. Triethylamine (544 μl, 3.903 mmol) was added thereinto and the mixture was heated at reflux with stirring. After confirming the completion of the reaction, the reaction solution was diluted in ethyl acetate, washed successively with 5% sodium bicarbonate, water and brine, and then evaporated under reduced pressure. The residue was column-chromatographed (hexane/ethyl acetate=4/1). (yield: 100%)

R_{f}=0.17 (hexane:ethyl acetate=4:1); IR(neat) 3538, 3437, 3113, 3084, 2967, 2935, 2879, 2449, 1913, 1736, 1592 cm⁻¹

### EXAMPLE 65

### Synthesis of Phenethyl-Thiocarbamate O-[1-(4-nitro-phenoxymethyl)-propyl]ester (28a) (R₄=4NO₂, R₅H)

60% NaH in oil (572.2 mg) was added into a dried flask, and the flask was filled with argon gas. A solution of 1-(4-nitro-phenoxy)-butan-2-ol (27a) (1.2086 g) in THF was added thereinto and stirred at 60° C. for 2 hours. At room temperature, phenethylisothiocyanate (17 ml) was added thereto and stirred. After confirming the completion of the reaction, the reaction mixture was extracted with ethyl acetate. The organic layer was washed with water and brine, and then evaporated under reduced pressure. The residue was column-chromatographed (hexane/ethyl acetate=15/1). (yield: 24.3%)

R_{f}=0.9/2.7=0.33 (hexane:ethyl acetate=4:1); MS (EI) 374(M⁺)

Compounds 28b˜g were synthesized according to the similar procedure as Example 65, and parts of spectral data thereof are shown below.

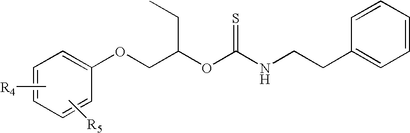

| Examples | Compounds | R₄ | R₅ | Spectral data |
|---|---|---|---|---|
| 66 | 28b | 4-OCH₃ | H | ¹H NMR(300MHz, CDCl₃): 7.25-7.06(m, 5H), |
| | | | | 6.82-6.73(m, 4H), 6.55-6.53(m, 2/5H), |
| | | | | 6.23-6.18(m, 3/5H), 5.64-5.56(m, 1H), |
| | | | | 4.05-3.94(m, 2H), 3.79-3.71(m, 6/5H), 3.70(s, |
| | | | | 6/5H), 3.69(s, 4/5H), 3.46-3.36(m, 4/5H), 2.86(t, |
| | | | | 6/5H, J=7.4), 2.73(t, 4/5H), 1.90-1.73(m, 2H), |
| | | | | 0.94(t, 6/5H, J=7.4Hz), 0.90(t, 9/5H) |
| 67 | 28c | 4-OBn | H | ¹H NMR(300MHz, CDCl₃): 7.46-7.28(m, 5H), |
| | | | | 6.95-6.85(m, 4H), 5.04(s, 2H), 3.98-3.92(m, 2H), |
| | | | | 3.83-3.77(m, 1H), 1.67-1.58(m, 2H), 1.05(t, 3H, |
| | | | | J=7.4Hz) |
| 68 | 28d | 4-CN | H | ¹H NMR(300MHz, CDCl₃): 7.35-7.21(m, 9H), |
| | | | | 6.60(s, 3/5H), 6.20(s, 2/5H), 3.87-3.81(q, 6/5H, |
| | | | | J=7.0Hz), 3.58-3.52(q, 4/5H, J=7.0Hz), |
| | | | | 2.97-2.93(t, 6/5H, J=7.0Hz), 2.86-2.81(t, 4/5H, |
| | | | | J=7.0Hz), 1.39-1.28(m, 5H) |
| 69 | 28e | 4-OH | 3-OCH₃ | ¹H NMR(300MHz, CDCl₃): 7.30-7.20(m, 5H), |
| | | | | 6.79(d.d, 1H, J=2.7, 8.7), 6.54(d, 1H, J=2.7), |
| | | | | 6.42-6.37(m, 1H), 5.66(s, 1/3H), 5.31(s, 2/3H), |
| | | | | 4.13-4.05(m, 3H), 4.83(m, 10/3H), 3.53-3.45(m, |
| | | | | 5/3H), 2.93(t, 4/3H, J=7.0), 2.80(t, 2/3H, J=7.0), |
| | | | | 1.96-1.79(m, 2H), 1.04-0.95(m, 3H) |
| 70 | 28f | H | 3-NH₂ | ¹H NMR(300MHz, CDCl₃): 7.29-7.11(5H, m), |
| | | | | 7.00(1H, t, J=8.0), 6.59(1/3H, s), 6.32-6.23(3H, |
| | | | | m), 6.32-6.23(2/3H, m), 5.67-5.60(1H, m), |
| | | | | 4.14-4.00(2H, m), 3.82-3.76(4/3H, m), 3.63(2H, |
| | | | | s), 3.50-3.43(2/3H, m), 2.90(4/3H, t, J=7.2), |
| | | | | 2.77(2/3H, t, J=7.2), 1.89(2/3H, d.q, J=7.2, 7.2), |
| | | | | 1.81(4/3H, d.q, J=7.2, 7.2), 0.98(1H, t, J=7.2), |
| | | | | 0.94(2H, t, J=7.2) |
| 71 | 28g | H | 3-NHSO₂CH₃ | ¹H NMR(400MHz, CDCl₃) 7.30-7.11(5H, m), |
| | | | | 6.83-6.81(1H, m), 6.76-6.70(2H, m), 6.65(1.3H, |
| | | | | s), 6.40(1H, s), 6.34(2/3H, s), 5.69-5.65(1H, m), |
| | | | | 4.15-4.12(1/3H, m), 4.10-4.08(2/3H, m), |
| | | | | 3.86-3.72(4/3H, m), 3.53-3.41(2/3H, m), 2.99(3H, |
| | | | | s), 2.96(1H, s), 2.91(4/3H, t, J=7.2), 2.78(2/3H, t, |
| | | | | J=7.2), 1.89(2/3H, d.q, J=7.6, 7.6), 1.81(4/3H, |
| | | | | d.q, J=7.6, 7.6), 0.99(1H, t, J=7.6), 0.95(2H, t, |
| | | | | J=7.6) |

### EXAMPLE 72

### Synthesis of 1-(4-amino-phenoxy)-butan-2-ol (29a)

1-(4-nitro-phenoxy)-butan-2-ol (27a) (530 mg) was poured into a flask and dissolved in methanol. Pd—C (53 mg) was poured thereinto and stirred at hydrogen gas atmosphere. The reaction mixture was filtered through cellite and the filtrate was evaporated under reduced pressure. The obtained residue was column-chromatographed (hexane/ethyl acetate=2/1).

R_{f}=0.14 (hexane:ethyl acetate=2:1); IR(neat) 3361, 2966, 2933, 2827, 1630, 1511, 1462, 1380, 1356, 1232 cm⁻¹

### EXAMPLE 73

### Synthesis of Phenethyl-Thiocarbamate O-[1-(4-amino-phenoxymethyl)-propyl]ester (30a) (R₄=H, R₅H)

The title compound was synthesized according to the procedure as the Example 4, using 1-(4-amino-phenoxy)-butan-2-ol (29) as a starting material.

R_{f}=0.26 (hexane:ethyl acetate=2:1); IR(neat) 3356, 3027, 2968, 2934, 2877, 2359, 1868, 1625, 1510, 1455 cm⁻¹

Compounds 30b˜e were synthesized according to the similar procedure as the Example 73, and parts of spectral data thereof are shown below.

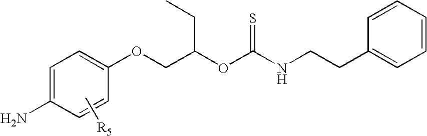

| Examples | Compounds | R₅ | Spectral data |
|---|---|---|---|
| 74 | 30b | 3-F | ¹H NMR(500MHz, CDCl₃) 7.35-7.16(5H, m), 6.74-6.58(3H, |
| | | | m), 6.74-6.58(1/3H, m), 6.33-6.31(2/3H, m), 5.69-5.66(1H, |
| | | | m), 4.12-4.02(1H, m), 3.87-3.80(2H, m), 3.47(2H, s), |
| | | | 2.97-2.92(4/3H, m), 2.84-2.82(2/3H, m), 1.92(2/3H, d.q, |
| | | | J=7.3, 7.3), 1.85(4/3H, d.q, J=7.3, 7.3), 1.07-0.96(3H, m) |
| 75 | 30c | 3-CH₃ | ¹H NMR(400MHz, CDCl₃) 7.30-7.12(5H, m), |
| | | | 6.70-6.61(2H ,m), 6.60-6.53(1H, m), 6.60-6.53(1/3H, m), |
| | | | 6.27(2/3H, d, J=4.0Hz), 3.88-3.69(3H, m), 3.66-3.43(2H, m), |
| | | | 3.31(2H, s), 2.91-2.83(4/3H, m), 2.80-2.74(2/3H, m), 2.11(3H, |
| | | | d, J=3.2), 1.81-1.54(2H, m), 0.94(2H, t, J=7.2), 0.89(1H, d.t, |
| | | | J=2.8, 7.2Hz) |
| 76 | 30d | 3-CF₃ | ¹H NMR(400MHz, CDCl₃) 8.00(1H, d.d, J=4.0, 9.2Hz), |
| | | | 7.32(1H, d, J=3.1), 7.13(1H, d, J=3.1, 8.4Hz), 4.08(1H, s), |
| | | | 3.97(2H, s), 1.66-1.62(2H, m), 1.04(3H, d.t, J=4.4, 7.2Hz) |
| 77 | 30e | 3-Cl | ¹H NMR(400MHz, CDCl₃) δ7.30-7.12(5H, m), 6.86(1H, d.d, |
| | | | J=3.2, 6.8), 6.68(2H, s), 5.66-5.61(1H, m), 4.03(4/3H, d.d, |
| | | | J=4.1, 10.4), 3.97(2/3H, d.d, J=4.1, 10.4), 3.84-3.77(4/3H, m), |
| | | | 3.50-3.43(2/3H, m), 2.90(4/3H, t, J=7.2), 2.77(2/3H, t, |
| | | | J=7.2), 1.87(2/3H, d.q, J=7.2), 1.80(4/3H, d.q, J=7.2), |
| | | | 0.98(1H, t, J=7.2), 0.94(2H, t, J=7.2) |

### EXAMPLE 78

### Synthesis of [4-(2-phenethylthiocarbamoyloxy-butoxy)-phenyl]-carbamate methyl ester (31a) (R=OCOCH₃, R₅H)

Phenethyl-thiocarbamate O-[1-(4-amino-phenoxymethyl)-propyl]ester 30 (15.5 mg) was dissolved in methylene chloride and the solution poured into a dried flask filled with argon gas, followed by adding methylchloroformate (5.8 μl) and pyridine (3.6 μl) thereinto and then stirring for 2 hours. The reaction solution was diluted in methylene chloride, washed successively with 5% CuSO₄, water and brine, and then evaporated under reduced pressure. The obtained residue was column-chromatographed (hexane/ethyl acetate=12/1).(yield: 58.0%)

R_{f}=0.61 (hexane:ethyl acetate=2:1); IR(neat) 3316, 3027, 2969, 2936, 2878, 2360, 1736, 1601, 1512, 1455 cm⁻¹

Compounds 31b˜g were synthesized according to the similar procedure as the Example 78, and parts of spectral data thereof are shown below.

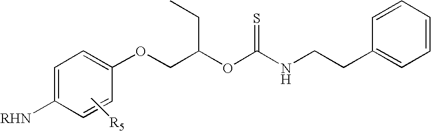

| Examples | Compounds | R | R₅ | Spectral data |
|---|---|---|---|---|
| 79 | 31b | SO₂CH₃ | H | IR (neat) 3590, 3271, 3061, 3027, 2969, 2934, 2878, |
| | | | | 2360, 1884, 1735, 1606, 1508, 1154 |
| 80 | 31c | COCH₃ | 3-F | ¹H NMR(400MHz, CDCl₃) 8.06-7.98(1H, m), |
| | | | | 7.28-7.04(5H, m), 6.72-6.67(2H, m), 6.61(1/3H, s), |
| | | | | 6.29(2/3H, s), 5.65(1H, m), 4.09-4.01(2H, m), |
| | | | | 3.81-3.75(2/3H, m), 3.51-3.45(2/3H, m), 2.91(4/3H, t, |
| | | | | J=9.6), 2.17(3H, s), 1.90-1.77(4/3H, m), |
| | | | | 1.76-1.65(2/3H, m), 1.00-0.95(3H, m) |
| 81 | 31d | SO₂CH₃ | 3-F | ¹H NMR(400MHz, CDCl₃) δ 7.43(1H, m), |
| | | | | 7.32-7.12(5H, m), 6.80-6.75(1/3H, m), 6.30(1H, s), |
| | | | | 6.30-6.25(2/3H, m), 5.70(1H, m), 4.17-4.08(2H, m), |
| | | | | 3.82(4/3H, d.q, J=7.2Hz), 3.52(2/3H, d.q, J=7.2Hz), |
| | | | | 2.98(3H, s), 2.97-2.91(4/3H, t, J=7.2), 2.98(3H, s), |
| | | | | 2.97-2.91(4/3H, t, 1=7.2Hz), 1.90-1.77(4/3H, t, |
| | | | | J=7.2Hz), 2.82(2/3H, t, J=7.2Hz), 1.90-1.77(4/3H, |
| | | | | m), 1.77-1.57(2/3H, m), 1.00-0.95(3H, m) |
| 82 | 31e | SO₂CH₃ | 3-CH₃ | ¹NMR (400MHz, CDCl₃) δ 7.31-7.11(6H, m), |
| | | | | 6.82-6.69(2H, m), 6.65(1/3H, m), 6.30-6.23(2/3H, m), |
| | | | | 6.01(1H, d, J=10.0), 5.69-5.63(1/3H, m), |
| | | | | 5.52-5.43(2/3H, m), 3.94-3.88(1H, m), 3.85-3.69(2H, |
| | | | | m), 3.63-3.51(4/3H, m), 2.95(3H, s), 2.30(3H, d, |
| | | | | J=6.0), 1.91-1.57(2H, m), 1.01-0.89(3H, m) |
| 83 | 31f | SO₂CH₃ | 3-CF₃ | ¹H NMR(400MHz, CDCl₃) δ 7.68(1H, d.d, J=3.2, |
| | | | | 8.8Hz), 7.32-7.07(5H, m), 6.69(1/3H, t, J=6.0), |
| | | | | 6.42(1H, s), 6.28(2/3H, t, J=6.0Hz), 5.70-5.65(1H, |
| | | | | m), 4.20-4.07(2H, m), 3.86-3.74(4/3H, m), |
| | | | | 3.52-3.47(2/3H, m), 2.92(3H, s), 2.93-2.90(4/3H, m), |
| | | | | 2.81-2.77(2/3H, m), 1.89(2/3H, d.q, J=7.2Hz), |
| | | | | 1.82(4/3H, d.q, J=7.2Hz), 1.00(1H, t, J=7.2Hz), |
| | | | | 0.96(2H, t, J=7.2Hz) |
| 84 | 31g | SO₂CH₃ | 3-Cl | ¹H NMR(400MHz, CDCl₃) δ 7.51(1H, d.d, J=1.6, |
| | | | | 8.4), 7.28(2H, d.t, J=0.8, 7.6), 7.24-7.14(3H, m), |
| | | | | 7.01-6.99(1H, m), 6.87-6.83(1H, m), 6.72(1/3H, t, |
| | | | | J=5.6), 6.49(1H, s), 6.29(2/3H, t, J=5.6), 5.63(1H, m), |
| | | | | 4.12-4.03(2H, m), 3.80(4/3H, m), 3.49(2/3H, m), |
| | | | | 2.92(3H, s), 2.92(4/3H, t, J=7.2), 1.87(2/3H, d.q, |
| | | | | J=7.2), 1.81(4/3H, d.q, J=7.2), 0.99(1H, t, J=7.6), |
| | | | | 0.95(2H, t, J=7.6) |

### EXAMPLE 85

### Synthesis of 5-[4-(t-butyl-dimethyl-silanyloxy)-3-methoxy-phenyl]-3-ethyl-pent-2-enic Acid Ethyl Ester (34)

60% NaH (44.4 mg, 1.11 mmol) was dissolved in THF and triethylphosphonoacetate (0.22 ml, 1.11 mmol) was diluted in the solution. The diluted solution was poured into a flask and stirred for 30 minutes. To the mixture was added a solution of 1-[4-(t-butyl-dimethyl-silanyloxy)-3-methoxy-phenyl]-pentan-3-on in THF and stirred for 30 hours. Upon the completion of the reaction, the reaction was quenched by addition of a small amount of water, and the reaction mixture was extracted with ethyl acetate. The obtained organic layer was washed with water and saturated aqueous sodium chloride solution, and then evaporated under reduced pressure. The obtained residue was column-chromatographed (n-hexane/ethyl acetate—100/1) to yield the compound (57.1 mg) as an oil.

IR (neat) 2967, 2854, 1715, 1644, 1514, 1464, 1284, 1158 cm⁻¹; ¹H NMR (300 MHz, CDCl₃) 6.64-6.44(3H, m), 5.51(2/5H, s), 5.47(3/5H, s), 4.03-3.99(2H, m), 3.66(2/5H, s), 3.65(3/5H, s), 2.76-2.70(4/5H, m), 2.59-2.50(2H, m), (2.50-2.47(4/5H, m), 2.31-2.26(6/5H, m), 2.02-1.95(4/5H, qd, J=7.4, 1.4), 1.16(6/5H, t, J=3.6), 1.12(9/5H, t, J=7.4), 0.95(9/5H, t, J=7.4), 0.91(6/5H, t, J=7.4), 0.85(9H, s) 0.00(6H, s)

### EXAMPLE 86

### Synthesis of 5-[4-(t-butyl-dimethyl-silanyloxy)-3-methoxyphenyl]-3-ethyl-pentanic Acid Ethyl Ester (35)

5-[4-(t-Butyl-dimethyl-silanyloxy)-3-methoxy-phenyl]-3-ethyl-pent-2-enic acid ethyl ester (34) (156.7 mg, 0.40 mmol) was diluted in appropriate amount of methanol, and reduced under hydrogen atmosphere in the presence of palladium/carbon as a catalyst. The reaction mixture was stirred vigorously, and after 1 hour, the completion of the reaction was confirmed using TLC. Then, the reaction mixture was filtered and evapoarated under reduced pressure. The residue was column-chromatographed (n-hexane/ethyl acetate=15/1) to yield the pure compound (126 mg, 81%) as a pale yellow liquid.

¹H NMR (300 MHz, CDCl₃) 6.61(1H, d, J=8.0) 6.52(1H, d, J=2.0) 6.47(1H, dd, J=8.0, 2.0) 4.00(2H, q, J=7.1) 3.65(3H, s) 2.40(2H, t, J=8.2) 2.14(2H, d, J=6.5) 1.73(1H, septet, J=6.5) 1.51-1.39(2H, m) 1.32-1.20(2H, m) 1.11(3H, t, J=7.1) 0.85(9H, s) 0.76(3H, t, J=7.4) 0.00(6H, s); IR (neat) 3039, 2932, 2966, 1715, 1644, 1514, 1284, 1158 cm⁻¹.

### EXAMPLE 87

### Synthesis of 5-[4-(t-butyl-dimethyl-silanyloxy)-3-methoxy-phenyl-3-ethyl-pentanic Acid Phenethyl Amide (36)

Phenethylamine (0.16 ml, 1.26 mmol) was added into a flask and diluted with methylene chloride. At room temperature, to the diluted solution was slowly added trimethyl aluminum (2M solution, 0.63 ml) and stirred for 15 min. To the resulting mixture was added a solution of 5-[4(t-butyl-dimethyl-silanyloxy)-3-methoxyphenyl]-3-ethyl-pentanic acid ethyl ester (36) (246.7 ml, 0.63 mmol) diluted in methylene chloride, and the mixture was heated at reflux. After the confirming the completion of the reaction, to the reaction mixture was carefully added a small amount of diluted hydrochloric acid, and the mixture was extracted with methylene chloride. The organic layer was washed with water and saturated aqueous sodium chloride solution, and then evaporated under reduced pressure. The residue was column-chromatographed (n-hexane/ethyl acetate=6/1) to yield the compound (310 mg) as a colorless liquid.

IR (neat) 3421, 3297, 2930, 2857, 1643, 1514, 1463, 1283, 1157, 1126 cm⁻¹; ¹H NMR (300 MHz, CDCl₃) 7.15-7.01(5H, m), 6.60-6.57(1H, m), 6.52-6.40(2H, m), 3.80(1H, br).

### EXAMPLE 88

### Synthesis of 3-ethyl-5-(4-hydroxy-3-methoxy-phenyl)-pentanic Acid Phenethyl Amide (37a)

5-[4-(t-Butyl-dimethyl-silanyloxy)-3-methoxy-phenyl-3-ethyl-pentanic acid phenethyl amide (36) (149.8 mg, 0.32 mmol) was dissolved in THF. To the solution was slowly added tetrabutylammonium fluoride (1M solution, 0.7 ml, 0.7 mmol) thereto and stirred for 15 min, followed by confirming the completion of the reaction using TLC. The reaction mixture was extracted with ethyl acetate. The organic layer was washed successively with water and saturated aqueous sodium chloride solution, dried over anhydrous Na₂SO₄, and then concentrated under reduced pressure. The residue was column-chromatographed (n-hexane/ethyl acetate=7/1, SiO₂) to yield thee pure compound (32 mg, 28%) as a colorless oil.

IR (neat) 3536, 3299, 3025, 2934, 2858, 1644, 1516, 1454 cm⁻¹; ¹H NMR (300 MHz, CDCl₃) 7.14-6.98(5H, m), 6.64(1H, d, J=7.9), F6.49 (1H, d, J=1.7), 6.46(1H, dd, J=7.9, 1.7), 5.42(1H, b), 5.28(1H, b), 3.69(3H, s), 3.34(2H, q, J=6.5), 2.62(2H, t, J=6.9), 2.35(2H, m), 1.91(2H, d, J=6.9), 1.68(1H, quin, J=6.5), 1.42-1.34(2H, m), 1.25-1.14(2H, m), 0.70(3H, t, J=7.4); HR-CI MS Obsd, m/z 356.2222; Calcd for C₂₂H₃₀NO₃, m/z 356.2226 (M⁺+H).

### EXAMPLE 89

### Synthesis of 8-[4(t-butyl-dimethyl-silanyloxy)-3-methoxy-phenyl]-6-ethyl-1-phenyl-octan-4-thi one (38)

Lawesson's reagent was diluted in toluene. To the diluted solution was added 3-ethyl-5-(4-hydroxy-3-methoxy-phenyl)-pentanic acid phenethyl amide (36) (160.3 mg, 0.34 mmol), and the mixture was heated at reflux and then cooled. After the completion of the reaction, the toluene was removed therefrom using a pressure-reducing distillatory apparatus. The reaction mixture was extracted with hexane. The organic layer was washed with water and saturated aqueous sodium chloride solution, and then evaporated under reduced pressure. The obtained mixture was column-chromatographed (n-hexane/ethyl acetate=12/1) to yield the compound (77.3 mg) as a pale yellow liquid.

R_{f}=0.38 (n-hexane/ethyl acetate=6/1); IR (neat) 3357, 3246, 3027, 2929, 2850, 1514, 1455, 1411, 1282, 1151 cm⁻¹; ¹H NMR (300 MHz, CDCl₃) 7.27-7.04(5H, m) 6.89(1H, s) 6.59(1H, d, J=8.0) 6.52(1H, d, J=1.8) 6.44(1H, dd, J=8.0, 1.8) 3.80(2H, q, J=6.5) 3.65(3H, s) 2.81(2H, t, J=7.0) 2.45-2.34(4H, m) 1.54-1.38(211, m) 1.29-1.18(2H, m) 0.85(9H, s) 0.71(3H, t, J=7.3) 0.00(6H, s).

### EXAMPLE 90

### Synthesis of 3-ethyl-5-(4-hydroxy-3-methoxy-phenyl)-pentanethioic Acid Phenethyl Amide (37b)

8-[4-(t-Butyl-dimethyl-silanyloxy)-3-methoxy-phenyl]-6-ethyl-1-phenyl-octane-4-thione (38) (77.3 mg, 0.16 mmol) was dissolved in THF, and to the solution was slowly added tetrabutylammonium fluoride (1M solution, 0.4 ml, 0.4 mmol), followed by stirring for 15 min and confirming the completion of the reaction. The reaction mixture was extracted with ethyl acetate. The organic layer was washed successively with water and saturated aqueous sodium chloride solution, dried over anhydrous Na₂SO₄, and then concentrated under reduced pressure. The obtained residue was column-chromatographed (n-hexane/ethyl acetate=5/1, SiO₂) to yield the pure compound (32.7 mg, 55%) as a colorless oil.

IR (neat) 3524, 3307, 3024, 2934, 2858, 1603, 1514, 1455 cm⁻¹; ¹H NMR (300 MHz, CDCl₃) 7.34-7.20(5H, m), 7.08(1H, s), 6.83(1H, d, J=8.0), 6.70(1H, d, J=1.9), 6.65(1H, dd, J=8.0, 1.9), 5.49(1H, s), 3.99-3.92(2H, m), 3.89(3H, s), 2.97(2H, t, J=6.9), 2.63-2.47(4H, m), 2.03(1H, qin, J=6.5), 1.57(2H, q, J=6.4), 1.37(2H, qin, J7.1), 0.87(3H, t, J=7.4); HR-CI MS Obsd, m/z 372.2007 Calcd for C₂₂H₃₀NO₂S, m/z 372.1998 (M⁺+H).

### EXAMPLE 91

### Synthesis of N-(4-iodophenyl)-methanesulfonamide (40a) (R₄=4-methanesulfonamide, R₅=Hydrogen, X=I)

4-Iodoaniline (200 mg) was dissolved in dichloromethane (2 ml), and to the solution were added pyridine (140 μl) and methanesulfonyl chloride (0.1 ml), followed by stirring at room temperature for 1 hour. After the addition of 1M hydrochloric acid, the reaction mixture was diluted with ethyl acetate (30 ml). The organic layer was washed successively with water and saturated aqueous sodium chloride solution, dried over magnesium sulfate, and then filtered. The filtrate was concentrated under reduced pressure, and the residue was chromatographed on a silica gel column eluting with ethyl acetate/hexane (1/4) to yield the compound 40a (252 mg, 85%).

¹H-NMR(300 MHz, CDCl₃) δ 7.64(d, 2H, J8.8 Hz), 7.98(d, 2H, J=8.8 Hz), 6.91(s, 1H), 2.30(s, 3H).

### EXAMPLE 92

### Synthesis of 5-phenylpentyn-3-ol (41)

3-Phenyl-1-propanol (1.58 g) was dissolved in dichloromethane (15 ml), and to the solution was added 4 angstrom of molecular sieve. To an ice-cold of the mixture was added pyridinium dichromate (6.1 g). The reaction mixture was stirred at room temperature for 3 hours, diluted with ether, and then filtered. The filtrate was concentrated under reduced pressure, and the residue was column-chromatographed to yield the aldehyde. The obtained aldehyde was dissolved in ether (10 ml), and to the solution was added dropwise 0.5M ethynylmagnesium bromide (15 ml), followed by stirring for 1 hour. To the mixture was added aqueous ammonium chloride solution to quench the reaction. Then, the reaction mixture was diluted with ethyl acetate (60 ml), washed successively with water and saturated aqueous sodium chloride solution, dried over magnesium sulfate, and then filtered. The filtrate was concentrated under reduced pressure, and the obtained residue was chromatographed on a silica gel column eluting with ethyl acetate/hexane (1/5) to yield the compound 41 (879 mg, 29%/).

¹H-NMR(300 MHz, CDCl₃): δ 7.19-7.33(m, 5H), 4.38(dt, 1H, J=6.6 Hz), 2.82(t, 2H, J=8 Hz), 2.51(d, 1H, J=2.2 Hz), 2.01-2.09(m, 2H).

### EXAMPLE 93

### Synthesis of N-(3-hydroxy-5-phenylpentynylphenyl)methanesulfonamide (42a) (R₄=4-methanesulfonamide, R₅=Hydrogen)

Compound 40 (252 mg) prepared according to the procedure as described in Example 91 was dissolved in diethyl amine (2 ml) and pyridine (1 ml). To the solution was added phenethyl propargylalcohol 41 (136 mg) prepared according to the procedure as described in Example 92, tetrakistriphenyl phosphine (49 mg), copper iodide (16 mg) and triphenyl phosphine (22 mg) and then refluxed for 18 hours. After cooling, the reaction mixture was diluted with ether and filtered through cellite. The filtrate was concentrated under reduced pressure, and the residue was chromatographed on a silica gel column eluting with ethyl acetate/hexane (1/3) to yield the compound 42a (200 mg, 72%).

¹H-NMR(300 MHz, CDCl₃): δ 7.34(d, 2H, J=8.8 Hz), 7.08-7.25(m, 51H), 6.93(s, 1H), 4.53(q, 1H, J=0.7), 2.96(s, 3H), 2.78(t, 2H, J=8.0 Hz), 2.01-2.11(m, 2H).

### EXAMPLE 94

### Synthesis of N-(3-hydroxy-5-phenylpentylphenyl)methanesulfonamide (43a) (R₄=4-methanesulfonamide, R₅=Hydrogen)

Compound 42a (200 mg) prepared according to the procedure as described in Example 93 was dissolved in anhydrous methanol (4 ml), and to the solution was added a catalytic amount of 10% palladium/carbon, followed by filling the reactor with hydrogen gas. After stirring at room temperature for 2 hours, the reaction mixture was diluted with ether and filtered through cellite. The filtrate was concentrated under reduced pressure, and the residue was chromatographed on a silica gel column eluting with ethyl acetate/hexane (1/2) to yield the compound 43a (206 mg, 98%).

¹H-NMR(300 MHz, CDCl₃): δ 7.04-7.23(m, 91), 6.66(s, 1M), 3.59(m, 1H), 2.91(s, 3H), 2.48-2.77(m, 4H), 1.70-1.77(m, 4H).

### EXAMPLE 95

### Synthesis of Phenethylthiocarbamic Acid (Methanesulfonylaminophenylethyl)propylethyl Ester (44a) (R₄4-methanesulfonamide, R₅=Hydrogen)

Compound 43a (27 mg) prepared according to the procedure as described in Example 94 was dissolved in tetrahydrofuran (2 ml). To an ice-cold of the solution was added 95% sodium hydride (16 mg) and phenethyl isothiocyanate (40 μl) successively, followed by stirring at 40° C. for 4 hours. To the reaction solution was added aqueous ammonium chloride to quench the reaction. The reaction mixture was diluted with ethyl acetate (20 ml), washed with water and saturated aqueous sodium chloride solution, dried over magnesium sulfate and then filtered. The filtrate was concentrated under reduced pressure, and the residue was chromatographed on a silica gel column eluting with ethyl acetate/hexane (1/3) to yield the compound 44a (27 mg, 67%).

¹H-NMR(300 MHz, CDCl₃): δ 7.12-7.33(m, 14H), 6.69(t, 1/3H), 6.12(t, 2/3H) 6.60(s, 1H), 5.56(m, 1H), 3.80(q, 2H, J=12.7 Hz), 2.96(d, 3H, J=2.8 Hz), 2.93(t, 4/3H, J=7.0), 2.80(t, 2/3H, J=7.0), 2.59-2.669(m, 4H), 1.86-2.03(m, 4H).

### EXAMPLE 96

### Synthesis of Phenylcarbamic Acid (Methanesulfonaminophenylethyl)phenylpropyl Ester (45a)

Compound 43a (23 mg) prepared according to the procedure as described in Example 94 was dissolved in benzene (1.5 ml), and to the solution was added phenethyl isocyanate (40 μl), followed by refluxing for 4 hours. The reaction mixture was concentrated under reduced pressure, and the residue was chromatographed on a silica gel column eluting with ethyl acetate/hexane (1/3) to yield the compound 45a (27 mg, 81%o).

¹H-NMR(300 MHz, CDCl₃): a 7.07-7.26(m, 14H), 6.74(s, 1H), 4.95(m, 1H), 4.61(t, 11I), 3.30-3.40(m, 2H), 2.89(s, 3H), 2.76(t, 2H, J=6.8), 2.50-2.57(m, 41), 1.77(m, 4H).

### EXAMPLE 97

### Synthesis of 4-iodo-benzenesulfonamide (40b)

Pipsyl chloride (100 mg) was dissolved in 28% ammonia solution 4 ml), followed by stirring at room temperature for 1 hour. The reaction mixture was extracted with ethyl acetate (20 ml). The organic layer was washed with water and saturared aqueous sodium chloride solution, dried over magnesium sulfate. The filtrate was concentrated under reduced pressure, and the residue was chromatographed on a silica gel column eluting with ethyl acetate/hexane (1/2) to yield the compound 40b (89 mg, 100%).

¹H-NMR(300 MHz, CD₃OD): δ 7.91(td, 1H, J=9.0 Hz), 7.63(td, 1H, J=9.0 Hz).

### EXAMPLE 98

### Synthesis of Phenethylcarbamic Acid 3-phenyl-1-(4-sulfamoylphenylethyl)propyl Ester (45b) (R₄=Aminosulfonyl, R₅=Hydrogen)

Compound 45b was synthesized according to the procedures as described in Examples 93, 94 and 96 and Scheme 9 using compound 40b as a starting material. (yield: 8%)

¹H-NMR(300 MHz, CDCl₃): δ 7.75(d, 2H, J=8.3 Hz), 7.07-7.26(m, 12H), 4.73(m, 3H), 4.57(t, 1H), 3.31-3.41(m, 2H), 2.69-2.78(m, 2H), 2.52-2.62(m, 4H), 1.80(m, 4H).

### EXAMPLE 99

### Synthesis of 4-bromo-2-Hydroxybenzoic Acid Methylester 40c-1) (R₄=4-methoxycarbonyl, R₅=3-hydroxy)

3-Bromophenol (1 g) was dissolved in 50% aqueous sodium hydroxide solution (5 ml), and to the solution added powdered copper (30 mg) and carbon tetrachloride (0.8 ml), followed by refluxing for 17 hours. The resulting mixture was acidified with a concentrated hydrochloric acid, and extracted with ethyl acetate. The obtained organic layer was washed with saturated aqueous sodium chloride solution, dried over magnesium sulfate and then filtered. The filtrate was concentrated under reduced pressure, and the residue was dissolved in ether, followed by adding diazomethane to terminate the reaction. The reaction mixture was concentrated under reduced pressure, and the obtained residue was chromatographed on a silica gel column eluting with ethyl acetate/hexane (1/5) to yield the compound 40c-1 (62 mg, 5%).

¹H-NMR(300 MHz, CDCl₃): δ 10.76(s, 1H), 7.11(d, 1H, J=1.7 Hz), 6.95(dd, 1H, J=8.5 Hz), 3.88(s, 3H)

### EXAMPLE 100

### Synthesis of 4-bromo-2-methoxymethoxybenzoic Acid Methylester (40c-2) (R₄=4-methoxycarbonyl, R₅=3-methyloxymethoxy)

Compound 40c-1 (62 mg) prepared according to the procedure as described in Example 99 was dissolved in tetrahydrofuran (2 ml). To the solution was added 60% sodium hydride (27 mg) in ice-cold bath, and added chloromethyl methyl ether (30 μl) at room temperature and then stirred for 1 hour. To the reaction solution was added aqueous ammonium chloride solution to quench the reaction. The reaction mixture was diluted with ethyl acetate (30 ml), washed with water and saturated aqueous sodium chloride solution, dried over magnesium sulfate and then filtered. The filtrate was concentrated under reduced pressure, and the residue was chromatographed on a silica gel column eluting with ethyl acetate/hexane (1/4) to yield the compound 40c-2 (63 mg, 85%).

¹H-NMR(300 MHz, CDCl₃): δ 7.30(d, 1H, J=8.3 Hz), 7.42(d, 1H, J=2.0 Hz), 7.22(dd, 1H, J=8.3 Hz), 5.28(s, 2H), 3.92(s, 3H), 3.56(s, 3H).

### EXAMPLE 101

### Synthesis of 4-(3-hydroxy-5-phenylpentyl)-2-methoxymethoxybenzoic Acid Methylester (43c-1) (R₄=4-methoxycarbonyl, R₅=3-methyloxymethoxy)

Compound 43c-1 was synthesized according to the procedures as described in Examples 93 and 94 using compound 40c-2 as a starting material. (yield: 61%)

¹H-NMR(300 MHz, CDCl₃): δ 7.65(d, 1H, J=8.0 Hz), 7.09-7.24(m, 5H), 6.94(d, 1H, J=1.2 Hz), 6.80(dd, 1H, J=8.0 Hz), 5.17(s, 2H), 3.81(s, 3H), 3.54-3.62(m, 1H), 3.45(s, 3H), 2.55-2.82(m, 4H), 1.63-1.77(m, 4H).

### EXAMPLE 102

### Synthesis of 4-(3-hydroxy-5-phenylpentyl)-2-methoxymethoxybenzoic Acid (43c-2) (R₄=4-carboxyl, R₅=3-methyloxymethoxy)

Compound 43c-1 (50 mg) prepared according to the procedure as described in Example 101 was dissolved in a mixed solution (2 ml, 1:1) of tetrahydrofuran and water, and to the solution was added LiOH H₂O (30 mg), followed by stirring at room temperature for 17 hours. The resulting mixture was acidified with 1M hydrochloric acid, extracted with ethyl acetate. The organic layer was washed with water and saturated aqueous sodium chloride solution, dried over magnesium sulfate and then filtered. The filtrate was concentrated under reduced pressure, and the residue was chromatographed on a silica gel column eluting with dichloromethane/methanol (20/1) to yield the compound 43c-2 (28 mg, 58%).

¹H-NMR(400 MHz, CDCl₃): δ 8.10(d, 1H, J=8.0 Hz), 7.28-7.33(m, 2H), 7.20-7.23(m, 3H), 7.02(d, 1H, J=8.0 Hz), 5.42(s, 2H), 3.67-3.70(m, 1H), 3.58(s, 3H), 2.80-2.85(m, 2H), 2.70-2.75(m, 2H), 1.78-1.87(m, 41).

### EXAMPLE 103

### Synthesis of 2-methoxymethoxy-4-(3-phenethylthiocarbamoyloxy-5-phenylpentyl) Benzoic Acid (44c-1) (R₄=4-carboxyl, R₅=3-methyloxymethoxy)

Compound 44c-1 was synthesized according to the procedure as described in Examples 95 using compound 43c-2 as a starting material. (yield: 48%)

¹H-NMR(300 MHz, CDCl₃): δ 8.00(d, 1H, J=8.0 Hz), 7.06-7.27(m, 10H), 6.85-7.04(m, 2H), 6.62(t, 1/3H, J=5.9 Hz), 6.11(t, 2/3H, 5.9 Hz), 5.46-5.54(m, 1H), 5.24-5.37(m, 2H), 3.68-3.78(m, 2H), 3.50(s, 3H), 2.87(t, 4/3H, J=6.8 Hz), 2.74(t, 2/3H, J=6.8 Hz), 2.51-2.67(m, 4H), 1.75-2.04(m, 4H)

### EXAMPLE 104

### Synthesis of 2-hydroxy-4-(3-phenethylthiocarbamoyloxy-5-phenylpentyl) Benzoic Acid (44c-2) (R₄=4-carboxyl, R₅=3-hydroxy)

Compound 44c-1 (20 mg) prepared according to the procedure as described in Example 103 was dissolved in a dichloromethane (2 ml), and to the solution was added trifluoroacetic acid (20 μl), followed by stirring for 40 min. After being adjusted to pH 6 using an aqueous sodium bicarbonate solution, the reaction mixture was extracted with ethyl acetate. The organic layer was washed with saturated aqueous sodium chloride solution, dried over magnesium sulfate and then filtered. The filtrate was concentrated under reduced pressure, and the obtained residue was chromatographed on a silica gel column eluting with dichloromethane/methanol (15/1) to yield the compound 44c-2 (8 mg, 44%).

¹H-NMR(300 MHz, CD₃OD): δ 7.73(d, 1H, J=7.8 Hz), 7.10-7.28(m, 101), 6.45-6.68(m, 2H), 5.48-5.54(m, 1H), 3.70(t, 4/3H, J=8.0 Hz), 3.37(t, 2/3H, J=8.0 Hz), 2.92(t, 4/3H, J=7.1 Hz), 2.79(t, 2/3H, J=7.1 Hz), 2.61(m, 4H), 1.90(m, 4H).

### EXAMPLE 105

### Synthesis of 4-bromophthalic Acid Dimethyl Ester (40d) (R₄=4-methoxycarbonyl, R₅=3-methoxycarbonyl)

Sodium hydroxide (60 mg) was dissolved in water (25 ml), and to the solution was added 4-bromo-o-xylene (350 μl), followed by heating to 85° C. Potassium peimanganate (336 mg) was added thereto, and the mixture was refluxed for 3 hours and then cooled, followed by adding sodium sulfite (1.13 g) and then stirring for 20 min. The reaction mixture was filtered, acidified with concentrated hydrochloric acid, and extracted with ethyl acetate. The organic layer was conentrated under reduced pressure. The residue was dissolved in ether and to the solution was added diazomethane in ether to terminate the reaction. The reaction mixture was concentrated under reduced pressure. The residue was chromatographed on a silica gel column eluting with ethyl acetate/hexane (1/10) to yield the compound 40d (172 mg, 35%). ¹H-NMR(300 MHz, CDCl₃): δ 7.80(d, 1H, J=1.7 Hz), 7.58-7.66(m, 2H), 3.89(s, 3H), 3.87(s, 3H).

### EXAMPLE 106

### Synthesis of 4-(3-phenethylcarbamoyloxy-5-phenylpentyl) Phthalic Acid (44d)

Compound 44d was synthesized according to the procedures as described in Examples 93, 94, 95 and 102 using compound 40d as a starting material. (yield: 53%)

¹H-NMR(400 MHz, THF-d₈+D₂O): δ 7.15-7.29(m, 13H), 5.67(s, 1H), 3.68-3.74(m, 2H), 2.96(t, 2/31), 2.86(t, 1/3), 2.69(m, 41), 1.96(m, 4H).

### EXAMPLE 107

### Synthesis of Phenethylcarbamic Acid 1-[2-(3-fluoro-4-methanesulfonylamino-phenylethyl)-3-phenylpropyl Ester (45c) (R₄=4-methanesulfonyylamino, R₅=3-fluoro)

The title compound was synthesized according to the similar procedure as synthesizing method of compound 45a using 4-bromo-2-fluoroaniline as a starting material.

¹H-NMR(300 MHz, CDCl₃): δ 7.43(t, 1H, J=8.0 Hz), 7.13-7.32(m, 10H), 6.93(d, 2H, J=9.5 Hz), 4.83(m, 1H), 4.64(t, 1H), 3.37-3.48(m, 211, 2.98(s, 3H), 2.82(t, 2H, J=7.1 Hz), 2.58-2.62(m, 4H), 1.81-1.85(m, 4H).

Compounds 44e˜i were synthesized according to the similar procedure as synthesizing method of the compound 44a, and parts of spectral data thereof are shown below.

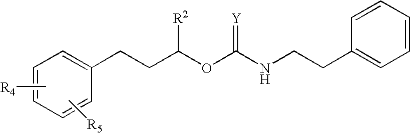

| Examples | Compounds | R₂ | R₄ | R₅ | Y | Spectral data |
|---|---|---|---|---|---|---|
| 108 | 44e | phenethyl | 4-H | 3-CO₂H | S | ¹H-NMR(300MHz, CDCl₃): δ |
| | | | | | | 7.93(d, 2H, J=8.1Hz), |
| | | | | | | 7.09-7.23(m, 11H), 6.58(t, 1/3H), |
| | | | | | | 6.02(t, 2/3H), 3.66-3.78(m, 2H), |
| | | | | | | 2.57-2.89(m, 6H), 1.78-2.06(m, |
| 4H) | | | | | | |
| 109 | 44f | C₂H₅ | 4-OH | 3-F | S | IR (neat) 3361, 3027, 2949, |
| | | | | | | 1604, 1519 MS(CI) m/z, |
| | | | | | | 362(M⁺+ 1), |
| 110 | 44g | C₂H₅ | 4-OH | 3-Cl | S | IR (neat) 3350, 3010; MS(CI) |
| | | | | | | m/z, 378(M⁺) |
| 111 | 44h | C₂H₅ | 4-OH | 3-NH₂ | S | IR(neat) 3340, 3010; MS(CI) |
| | | | | | | m/z, 358(M+) |
| 112 | 44i | C₂H₅ | 4-OH | 3-COOCH₃ | S | ¹H NMR(400MHz, CDCl₃): δ |
| | | | | | | 10.06(s, 1H, J=2.4Hz, OH), |
| | | | | | | 7.65(dd, 1H, J=2.4, 5.6Hz), |
| | | | | | | 7.34-7.21(m, 5H) 7.19(dd, 1H, |
| | | | | | | J=7.4, 9.0Hz), 6.91(dd, 1H, |
| | | | | | | J=3.6, 8.4Hz), 6.57(s, 1/3H, NH), |
| | | | | | | 6.16(s, 2/3H, NH), 5.48(septet, |
| | | | | | | 1H, J=6.0Hz), 3.94(d, 3H, |
| | | | | | | J=3.6Hz), 3.82(q, 4/3H, J=6.4Hz), |
| | | | | | | 3.55-3.45(m, 2/3H), 2.94(t, |
| | | | | | | 4/3H, J=7.2Hz), 2.82(t, 2/3H, |
| | | | | | | J=7.2Hz), 2.64-2.56(m, 2H), |
| | | | | | | 2.03-1.83(m, 2H), 1.74(quintet, |
| | | | | | | 2/3H, J=7.4Hz), 1.66(quintet, |
| | | | | | | 4/3H, J=7.4Hz), 0.95(t, 1H, |
| | | | | | | J=7.4Hz), 0.91(t, 2H, J=7.4Hz) |

### EXAMPLE 113

### Synthesis of 2-hydrxoy-5-(3-phenethylthiocarbamoyloxypentyl)-benzoic Acid (44j) (R₄=OH, R₅=3-CO₂H)

Compound 44i (13 mg) was dissolved completely in a mixed solution (3 ml, 3:1) of methanol and water, and to the solution was added lithium hydroxide (5 mg), followed by stirring at room temperature for 30 hours. The resulting mixture was concentrated under reduced pressure to remove the solvent, extracted with ethyl acetate (20 ml). The organic layer was washed with aqueous ammonium chloride solution (5 ml), water (5 ml) and saturated aqueous sodium chloride solution, and then dried over magnesium sulfate. The reaction mixture was concentrated under reduced pressure, and the obtained residue was column-chromatographed (dichloromethane/methanol 10/1) to yield the compound 44j (5 mg, 40%).

R_{f}=0.26 (dichloromethane:methanol=10:1); IR(NaCl): cm⁻¹ 3363, 3025, 2969, 2933, 2873, 1725, 1556, 1453, 1247, 1167, 830.

Compounds 46a˜f were synthesized according to the similar procedure as synthesizing method of the compound 44a, and parts of spectral data thereof are shown below.

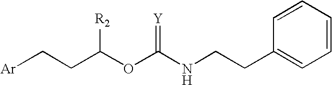

| Examples | Compounds | Ar | R₂ | Y | Spectral data |
|---|---|---|---|---|---|
| 114 | 46a | 6-carboxy- | phenethyl | S | ¹H-NMR(300MHz, CD₃OD): δ |
| | | pyridin-2-yl | | | 8.87(d, 1H, J=8.3Hz), 7.3(d, 1H, |
| | | | | | J=7.3Hz), 7.05-7.259(m, 11H), |
| | | | | | 5.38(m, 1H), 3.64-3.75(m, 2H), |
| | | | | | 2.74-2.92(m, 4H), 2.26-2.42(m, 2H), |
| | | | | | 1.35-1.68(m, 4H) |
| 115 | 46b | 6-amino- | ethyl | O | ¹H NMR(400MHz, CDCl₃): δ |
| | | pyridin-3-yl | | | 7.85(d, 1H, J=6.8Hz), 7.31-7.14(m, |
| | | | | | 6H), 6.42(dd, 1H, J=3.2, 8.4Hz), |
| | | | | | 5.44(q, 1H, J=6, 7.2Hz), 4.34(s, 2H, |
| | | | | | NH2), 3.79(q, 111, J=6.6Hz), |
| | | | | | 3.52-3.45(m, 1H), 2.92(t, 1H, |
| | | | | | J=7.0Hz), 2.80(t, 111, J=7.2Hz), |
| | | | | | 2.53-2.44(m, 1H), 1.86-1.76(m, 2H), |
| | | | | | 1.74-1.57(m, 2H), 8.94(dt, 3H, |
| | | | | | J=7.2, 16.8Hz) |
| 116 | 46c | 5-amino | ethyl | O | ¹H NMR(400MHz, CDCl₃): 0.86(t, |
| | | | | | 3H, J=7.2Hz), 1.23(s, 1H, NH), |
| | | | | | 1.55(t, 2H, J=6.8Hz), 1.84-1.86(m, |
| | | | | | 2H), 2.63-2.70(m, 2H), 2.79(t, 2H, |
| | | | | | J=6.8Hz), 3.40(d, 2H, J=6.8Hz), |
| | | | | | 3.44(s, 1H), 4.71(s, 2H, NH2), 6.89- |
| | | | | | 6.91(m 2H), 7.16-7.30(m, 5H), |
| | | | | | 7.99(s, 1H) |
| 117 | 46d | 5-methanesulf | ethyl | O | ¹H NMR(400MHz, CDCl₃): 0.89(t, |
| | | onylamino- | | | 3H, J=7.0Hz), 1.27(t, 3H, |
| | | pyridin-2-yl | | | J=7.2Hz), 1.58(m, 2H), 1.89(m, 2H), |
| | | | | | 2.05(s, 2H), 2.67-2.74(m, 2H), 2.82(t, |
| | | | | | 3H, J=6.6z), 3.01(s, 1H), 3.44(d, 2H, |
| | | | | | J=6.0Hz), 4.70-4.73(m, 2H), 6.93(d, |
| | | | | | 2H, J=2.4Hz), 7.20-7.33(m, 5H), |
| 118 | 46e | 6-methanesulf | ethyl | O | IR(NaCl/neat): cm⁻¹ 2123, 3026, |
| | | | | | 2928, 2857, 1734, 1681, 1556, 1496, |
| | | | | | 1354, 1247, 1173 |
| 119 | 46f | 1-t-butoxy-2 | ethyl | S | ¹H NMR(400MHz, CDCl₃): δ |
| | | | | | 8.20(s, 1H) 7.38-7.22(m, 5H), 7.07(s, |
| | | | | | 1H) 4.70-4.60(m, 1H, NH) |
| | | | | | 4.03-4.00(m, 1H) 3.74(t, 2H, |
| | | | | | J=7.2Hz) 3.04(td, 2H, J=6.8Hz, |
| | | | | | 25.6Hz), 2.78-2.51(m, 2H), |
| | | | | | 1.94-1.85(m, 2H), 1.62(sextet, 2H, |
| | | | | | J=7.2Hz), 1.50(d, 9H, J=8.8Hz), |
| | | | | | 0.92(t, 3H, J=7.6Hz) |

### EAXAMPLE 120

### Synthesis of Bromoindolecarboxylic Acid T-Butyl Ester (47)

5-Bromoindole (50 mg) was dissolved in dichloromethane (1.5 ml), and to the solution was added triethylanine (70 μl), dimethylaminopyridin (31 mg) and dibutyldicarbonate (85 mg), followed by stirring at room temperature for 1 hour. The reaction mixture was concentrated under reduced pressure, and the obtained residue was chromatographed on a silicagel column eluting with ethyl acetate/hexane (1/4) to yield the compound 47 (75 mg, 100%).

¹H-NMR(300 MHz, CDCl₃): δ 8.00(d, 1H, J=8.8 Hz), 7.66(d, 111, J=2.0 Hz), 7.57(d, 1H, J=3.7 Hz), 7.38(dd, 1H, J=8.8 Hz), 6.48(d, 1H, s-3.7 Hz), 1.65(s, 91).

### EAXAMPLE 121

### Synthesis of 5-(3-hydroxyphenylpentynyl)indolecarboxylic Acid T-Butyl Ester (48)

Compound 48 was synthesized according to the procedure as decribed in Example 93 using compound 47 as a starting material.(yield: 72%)

¹H-NMR(300 MHz, CDCl₃): δ 8.07(d, 1H, J=8.3 Hz), 7.64(d, 1H), 7.58(d, 1H, J=3.6 Hz), 7.36(dd, 1H, J=8, 5 Hz), 7.18-7.31(m, 5H), 6.52(d, 1H, J=3.7 Hz), 4.61(q, 1H), 2.88(t, 2H, J=8.0 Hz), 2.09-2.16(m, 2H), 1.88(d, 1H, f-5.6 Hz), 1.65(s, 9H).

### EAXAMPLE 122

### Synthesis of 5-(3-hydroxyphenylpentyl)indolecarboxylic Acid T-Butyl Ester (49)

Compound 49 was synthesized according to the procedure as decribed in Example 94 using compound 48 as a starting material.(yield: 84%)

¹H-NMR(300 MHz, CDCl₃): δ 8.02(d, 1H, f-8.0 Hz), 7.56(d, 1H, J=3.7 Hz), 7.35(d, 1H, J=1.0 Hz), 7.08-7.29(m, 6H), 6.49(d, 1H, J3.71z), 3.67(m, 1H), 2.56-2.95(m, 4H), 1.74-1.78(m, 4H).

### EAXAMPLE 123

### Synthesis of 5-(3-phenethylthiocarbamoyloxy-5-phenylpentyl)indolecarboxylic Acid T-Butyl Ester (50)

Compound 50 was synthesized according to the procedure as decribed in Example 95 using compound 49 as a starting material.(yield: 70%)

¹H-NMR(300 MHz, CDCl₃): δ 8.10(d, 1H, J=8.3 Hz), 7.54(d, 1H, f-3.7 Hz), 7.06-7.34(m, 12H), 6.49(d, 1H, J=3.9 Hz), 6.52(t, 1/3H), 6.07(t, 2/3H), 5.60-5.66(m, 1H), 3.78(q, 4/3H, J=6.5 Hz), 3.42(q, 2/3H, J=6.5 Hz), 2.90(t, 4/3H, 7.1 Hz), 2.78(t, 2/3H, J=7.1 Hz), 2.61-2.76(m, 4H), 1.91-2.12(m, 4H), 1.64(s, 9H).

### EXAMPLE 124

### Synthesis of Phenethylthiocarbamic Acid 2-indolethyl-3-phenylpropyl Ester (51)

Anhydrous compound 50 (10 mg) prepared according to the procedure as decribed in Example 123 was heated to 130˜140° C. under anhydrous condition for 30 min. The reaction mixture was chromatographed on a silicagel column eluting with ethyl acetate/hexane (1/4) to yield 4 mg of the compound 51. (yield: 61%)

¹H-NMR(300 MHz, CDCl₃): δ 8.01(s, 1H), 7.37(d, 1H, J=4.1 Hz), 7.08-7.27(m, 12H), 6.93-6.96(m, 1H), 6.48(t, 1/3H), 6.01(t, 2/3H), 6.42(t, 1H, J=2.2 Hz), 5.57(m, 1H), 3.73(q, 4/3H, J=13 Hz), 3.37(q, 2/3H, J=13 Hz), 2.85(t, 4/3H, J=-7.1 Hz), 2.75(t, 2/3H, J=7.1 Hz), 2.56-2.73(m, 41), 1.87-2.10(m, 4H).

### EXAMPLE 125

### Synthesis of 1-indole-5-phenylpentan-3-ol (52)

Compound 52 was synthesized according to the procedure as decribed in Example 124 using compound 51 as a starting material.(yield: 72%)

¹H-NMR(300 MHz, CDCl₃): δ 8.03(s, 1H), 738(s, 1H), 7.08-7.29(m, 7H), 6.97(dd, 1H, J=8.3 Hz), 6.41-6.43(m, 1H), 3.63(m, 1H), 2.542.86(m, 411, 1.69-1.83(m, 4H).

### EXAMPLE 126

### Synthesis of Phenethylcarbamic Acid 2-indolethyl-3-phenylpropyl Ester (53)

Compound 53 (yield: 59%) and 54 (yield: 24%) were synthesized according to the procedure as decribed in Example 96 using compound 52 as a starting material.

¹H NMR(300 MHz, CDCl₃): δ 8.02(s, 1H), 7.36(s, 1H), 7.08-7.26(m, 12H), 6.94(dd, 1H, J=8.3 Hz), 6.41-6.42(m, 1H), 4.85(m, 1H), 4.56(t, 1H), 3.35-3.44(m, 2H), 2.542.78(m, 61), 1.83(m, 4H).

### EXAMPLE 127

### Synthesis of 4-(3-hydroxy-5-phenyl-pent-1-enyl)-2-methoxyphenol (55)

Compound 3 (115 mg, 0.29 mmol) was diluted in tetrahydrofuran, and to the solution was slowly added tetrabutylammonium fluoride (1M solution in T=F, 0.72 ml), followed by string for 20 min. After confirming the completion of the reaction using TLC, the reaction mixture was extracted with ethyl acetate. The organic layer was washed successively with water (4 ml×2) and saturated saline solution (4 ml), dried over anhydrous Na₂SO₄, and then concentrated under reduced pressure. The residue was column-chromatographed (hexane/ethyl acetate=4/1) to yield the compound (81.7 mg, 99.6%) as a colorless oil.

IR(neat) 3440, 3025, 2938, 1718, 1674, 1597, 1514, 1454, 1271 cm⁻¹; ¹H NMR (300 MHz, CDCl₃): 7.25-7.07(5H, m), 6.83-6.80(3H), 6.43(1H, d, J=15.8), 6.01(1H, dd, J=15.8, 7.0), 5.58(1H, s), 4.21(1H, q, J=6.5), 3.84(3H, s), 2.72-2.65(2H, m), 1.94-1.84(2H, m), 1.54(1H, s); MS (EI) m/e(relative intensity) 284(M+) 266(47) 175(55) 137(83) 91(100).

### EXAMPLE 128

### Synthesis of 4(3-hydroxy-5-phenylpentyl)-2-methoxyphenol (56)

Compound 55 (46.1 mg, 0.16 mmol) was diluted in ethanol, and to the diluted solution was added palladium/carbon (20 mg), followed by stirring at hydrogen gas atmosphere. After confirming the completion of the reaction using TLC, the reaction mixture was filtered to remove Pd/C, and then concentrated under reduced pressure. The residue was column-chromatographed (hexane/ethyl acetate=4/1) to yield the compound (44.7 mg, 96.3%) as a colorless oil.

IR (neat) 3414, 3025, 2937, 1708, 1603, 1515, 1454, 1270, 1035 cm⁻¹; ¹H NMR (300 MHz, CDCl₃): 7.33-7.20(5H, m), 6.87-6.84(1H), 6.71-6.68(21H), 5.50(1H, s), 3.89(3H, s), 4.32(1H), 2.79-2.64(4H, m), 1.84-1.77(4H, m), 1.42(1H, d, J=5.2).

### EXAMPLE 129

### Synthesis of 4(3-hydroxy-5-phenylpentyl)-benzene-1,2-diol (57)

Compound 56 (198 mg, 0.61 mmol) was diluted in dichloroethane (10 ml). The diluted solution was poured, through cannula, into a branched flask which is filled with nitrogen gas, and then BBr₃ S(CH₃)₂ (1M solution in dissolved in 2.42 ml of hexane, 2.42 mmol) was slowly added thereto through an injector. After heating at reflux for 2 hours, the completion of the reaction was confirmed using TLC and to the solution was added H₂O (3 ml), followed by stirring for 10 min. The reaction solution was diluted with ether (50 ml), washed successively with H₂O (5 ml), 5% NaHCO₃ (5 ml), H₂O (5 ml×2) and saturated saline solution, dried over anhydrous Na₂SO₄, and then concentrated under reduced pressure. The residue was column-chromatographed (hexane/ethyl acetate 4/1, SiO₂) to yield the compound (187 mg, 98.8%) as a colorless oil.

R_{f}=0.41 (n-hexane: EtOAc=1:1, SiO₂); MS(CI) m/e 272(M⁺)

### EXAMPLE 130

### Synthesis of 1-(3,4-bis-methoxymethxoyphenyl)-5-phenylpentan-3-ol (58)

K₂CO₃ (1.4 g, 10.11 mmol) was poured into two-necked flask filled with nitrogen, and suspended in acetone (10 ml). A solution of compound 57(211 mg, 0.67 mmol) in acetone (2 ml) was poured thereinto through cannula and the mixture was stirred at 50° C. for 2 hours. Then, MOMCl (0.51 ml, 6.74 mmol) was added thereto, followed by stirring for 24 hours. After confirming the completion of the reaction using TLC, the reaction mixture was concentrated under reduced pressure to remove acetone, diluted with ethyl acetate (70 ml), washed successively with water (8 ml−2) and saturated saline solution (8 ml), dried over anhydrous sodium sulfate, and then concentrated under reduced pressure. The residue was column-chromatographed (hexane/ethyl acetate=12/1, SiO₂) to yield the compound (100 mg, 37.0%) as a colorless oil.

IR (neat) 3446, 3029, 2997, 1589, 1510 cm⁻¹; ¹H NMR (300 MHz, CDCl₃) 7.23-7.08(5H, m), 6.98(1H, d, J=8.2), 6.91(1H, d, J=2.0), 6.70(1H, dd, J=8.2, 2.0), 5.13(2H, s), 3.65-3.54(1H, m), 3.33(6H, s), 2.65-2.55(411 m), 1.74-1.65(4H, m)

### EXAMPLE 131

### Synthesis of Phenethylthiocarbamic Acid O-[3-(3,4-bis-methoxymethoxyphenyl)-1-phenethylpropyl] Ester (59)

Compound 58 (100 mg, 0.25 mmol) was diluted in tetrahydrofuran (8 ml). The diluted solution was poured, through cannula, into two-necked flask which is filled with nitrogen gas, and to the mixture was added sodium hydride (60% in mineral oil, 30 mg, 0.75 mmol), followed by stirring at reflux for 1 hour with the temperature being adjusted to 30° C. Phenethyl isothiocyanate (0.11 ml, 075 mmol) was slowly added dropwise thereto, and the mixture was stirred for 24 hours. After confirming the completion of the reaction using TLC, saturated ammonium chloride solution was added thereto to terminate the reaction. The reaction mixture was extracted with ethyl acetate (60 ml). The organic layer was washed with saturated ammonium chloride solution (7 ml), water (7 ml×3) and saturated saline solution (7 ml), dried over anhydrous sodium sulfate and then concentrated under reduced pressure. The obtained residue was column-chromatographed hexane/ethyl acetate=10/1, SiO₂) to yield the compound (72.8 mg, 51.7%) as a colorless oil.

IR (neat) 3310, 3025, 2951, 1589, 1511, 1454, 1259 cm⁻¹; ¹H NMR (300 MHz, CDCl₃) 7.27-7.01(10H, m), 6.98(1H, d, J=8.2), 6.90(1H, d, J=2.0), 6.69(1H, dd,J=8.2, 2.0), 6.47(1/3H, t, J=5.6), 6.07(2/3H, t, J=5.8), 5.59-5.48(1H, m), 5.17-5.11(4H, m), 3.74(4/311, q, J=6.7), 3.44(6H, s), 3.38(2/3H, q, J=6.7), 2.87(4/3H, t, J=7.1), 2.74(2/311, t, J=7.1), 2.65-2.50(4H, m), 2.06-1.81(4H, m).

### EXAMPLE 132

### Synthesis of Phenethylthiocarbamic Acid O-[3-(3,4-dihydroxyphenyl)-1-phenethylpropyl] Ester (60)

Compound 59 (51 mg, 0.09 mmol) was dissolved in a mixed solution of tetrahydrofuran (2 ml) and isopropanol (1 ml), and to the solution was slowly added a concentrated hydrochloric acid (0.2 ml), followed by stirring for 2 hours. After confirming disappearance of the reactant using TLC, the reaction mixture was concentrated under reduced pressure to remove the solvent, extracted with ethyl acetate (20 ml). The organic layer was washed successively with water (4 ml), saturated aqueous sodium bicarbonate solution (4 ml) and saturated saline solution (4 ml), dried over anhydrous sodium sulfate, and then concentrated under reduced pressure. The residue was column-chromatographed (hexane/ethyl acetate=3/1, SiO₂) to yield the compound (43 mg, 99.4%) as a colorless oil.

IR (neat) 3361, 3027, 2949, 1604, 1519 cm⁻¹; ¹H NMR (300 MHz, CDCl₃) 7.24-7.07(10H, m), 6.69(1/3H, d, J=8.0), 6.68(2/3H, d, J=8.0), 6.62(1/311, d, J=2.0), 6.60(2/3H, d, J=2.0), 6.53-6.50(4/3H, m), 6.03(2/3H, t, J=5.8), 5.52-5.44(4H, m), 5.35(1/3H, s), 5.26(2/3H, s), 5.11(1H, s), 3.74(4/31, —q, J=6.7), 3.34(2/3H, q, J=6.7), 2.86(4/3H, t, J=7.1), 2.71(2/3H, t, J=7.1), 2.60-2.43(4H, m), 1.97-1.82(4H, m).

In the below, it is confirmed by calcium influx test and electrophysiological test that the compounds of the present invention are antagonist to vanilloid receptor, further confirmed by analgesic effect test that they exhibit strong analgesic effects while showing no irritation, different from general agonists.

### EXPERIMENTAL EXAMPLE

### Biological Potency Test

(1) ⁴⁵ Ca Influx Test

1) Separation of Spinal Dorsal Root Ganglia (DRG) in Newborn Rats and Primary Culture Thereof

Neonatal (2-day old or younger than 2-day old) SD rats were put in ice for 5 minutes to anesthetize and disinfected with 70% ethanol. DRG of all part of spinal cord were dissected (Wood et al., 1988, J. Neurosci. 8, pp3208-3220) and collected in DME/F12 medium to which 1.2 g/l sodium bicarbonate, 50 mg/l gentamycin were added. The DRG were incubated sequentially at 37° C. for 30 min in 200 U/ml collagenase and 2.5 mg/ml trypsin, separately. The ganglia were washed twice with DME/F12 medium supplemented with 10% horse serum, triturated through a fire-polished Pasteur pipette, filtered through Nitex 40 membrane to obtain single cell suspension. This was subjected to centrifugation, then re-suspended in cell culture medium at certain level of cell density. As the cell culture medium, DME/F12 medium supplemented with 10% horse serum, diluted 1:1 with identical medium conditioned by C6 glioma cells (2 days on a confluent monolayer) was used, and NGF (Nerve Growth Factor) was added to final concentration of 200 ng/ml. After the cells were grown 2 days in medium where cytosine arabinoside (Ara-C, 100 i was added to kill dividing normeuronal cells, medium was changed to one without Ara-C. The resuspended cells were plated at a density of 1500-1700 neurons/well onto Terasaki plates previously coated with 10 μg/ml poly-D-ornithine.

2) ⁴⁵ Ca Influx Experiments

DRG nerve cells from the primary culture of 2-3 days were equilibrated by washing 4 times with BEPES (10 mM, pH 7.4)-buffered Ca 2+, Mg²⁺-free HBSS (H-HBSS). The solution in each well was removed from the individual well. Medium containing the test compound plus capsaicin (final concentration 0.5 μM) and ⁴⁵Ca (final concentration 10 μCi/ml) in H-HBSS was added to each well and incubated at room temperature for 10 min. Terasaki plates were washed six times with H-HBSS and dried in an oven. To each well, 0.3% SDS (10 μl) was added to elute ⁴⁵Ca. After the addition of 2 ml of scintillation cocktail into each well, the amount of ⁴⁵Ca influx into neuron was measured by counting radioactivity. Antagonistic activities of test compounds against vanilloid receptor were calculated as percent of the maximal response of capsaicin at a concentration of 0.5 μM and results are given as IC₅₀ (Table 1).

(2) Channel Activity Assay

Antagonistic activities of test compounds were assayed based on electrical change of cation channel connected to vanilloid receptor and experiments were conducted according to reference method (Oh et al., 1996, J. Neuroscience 16, pp1659-1667) (Table 1).

TABLE 1
Results of Calcium Influx and Patchclamp Test

| | Calcium Influx | Patch clamp Test |
|---|---|---|
| Examples | Test (IC₅₀) | (antagonistic activities) |
| 5 | 19.2 | + |
| 37 | 13.7 | |
| 39 | 20.9 | |
| 41 | 6.3 | |
| 43 | 9.9 | |
| 44 | 27.9 | |
| 51 | 25.0 | |
| 54 | 10.7 | + |
| 56 | 16.1 | + |
| 60 | 4.3 | |
| 63 | 9.8 | |
| 69 | 10.1 | |
| 71 | 17.6 | |
| 80 | 25.7 | |
| 81 | 25.7 | |
| 83 | 3.5 | + |
| 96 | 25.7 | |
| 108 | 18.8 | + |

NR: no response
+: antagonistic potency equal to capsazepine
++: antagonistic potency 10 times higher than capsazepine

(3) Analgesic Activity Test: Mouse Writhing Test by Inducing with Phenyl-p-quinone

Male ICR mice (mean body weight 25 g) were maintained in a controlled lighting environment (12 h on/12 h off) for experiment. Animals received an intraperitoneal injection of 0.3 ml of the chemical irritant phenyl-p-quinone (dissolved in saline containing 5% ethanol to be a dose of 4.5 mg/kg) and 6 min later, the number of abdominal constrictions was counted in the subsequent 6 min period. Animals (10 animals/group) received 0.2 ml of test compounds solution in vehicle of ethanol/Tween 80/saline (10/10/80) intraperitoneally 30 min before the injection of phenyl-p-quinone. A reduction in the number of writhes responding to the test drug compound relative to the number responding in saline control group was considered to be indicative of an analgesic effect. Analgesic effect was calculated by % inhibition equation (% inhibition=(C−T)/C×100), wherein C and T represent the number of writhes in control and compound-treated group, respectively (Table 2).

The test results demonstrated that analgesic effect of the compounds used in this experiment is potent, and in particular, it is significant to clarify that vanilloid receptor antagonist can exhibit such potent analgesic effect, and the results suggests that vanilloid receptor antagonist has potential as an analgesic agent.

TABLE 2
Test result of analgesic activity for writhing by phenyl-p-quinone

| | | Analgesic effect |
|---|---|---|
| Examples | Dose (mg/kg) | (% Inhibition) |
| 5 | 10 | 44 |
| 54 | 10 | 64 |
| 60 | 1 | 45 |

(4) Antiinflammatory Activity Test: TPA (12-O-tetradecanoylphorbol 13-acetate)-induced Mouse Ear Edema Test

Male ICR mice (body weight 25-30 g), 10 animals/group, were treated topically on the right ear with 30 μl of TPA (2.5 μg) solution in acetone and after 15 min, 30 td of acetone or test compound solution in acetone was applied topically. After six hours, an identical treatment was applied again. After twenty four hours following the treatment of TPA, the animals were sacrificed and ear tissue was dissected using 6 mm-diameter punch. Ear tissue dissected were weighed to the nearest 0.1 mg on an electrobalance. The increased weight of the tissue compared to control group was considered as an index of inflammation. The percent inhibition is defined by the following equation:

% inhibition=(C−T)/C×100, wherein C and T represent an increase of ear weight in TPA-treated and ThA+drug-treated group, respectively.

The above experiment shows that vanilloid receptor antagonist exhibits significant anti-inflammatory effects. This phenomenon can be understood by connecting with the action of vanilloid receptor in neurogenic inflammation, and suggests potential applicability of vanilloid receptor antagonist in various inflammatory diseases, in particular, neurogenic inflammatory diseases.

TABLE 3
TPA-induced mice ear edema test

| | | Anti-inflammatory effect |
|---|---|---|
| Examples | Dose (mg/ear) | (% Inhibition) |
| 5 | 1 | 33 |
| 54 | 1 | 40 |
| 60 | 1 | 45 |

### INDUSTRIAL APPLICABILITY

The compounds according to the present invention are useful in the prevention or treatment of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma and chronic obstructive pulmonary diseases, irritation in skin, eye or mucous membrane, fervescence, stomach-duodenal ulcer, inflammatory bowel disease, inflammatory disease, etc.

1. A compound of formula I: 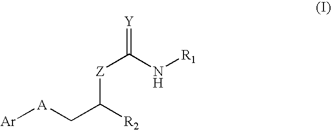
or a pharmaceutically acceptable salt thereof,
wherein,
R₁ represents Ar′—(CH₂)ₘ— (wherein Ar′ is phenyl, pyridinyl, thiophenyl or naphthalenyl substituted or unsubstituted with halogen or lower alkyl having 1 to 5 carbon atoms; or trifluoromethylphenyl, and m is 1, 2, 3 or 4), —(CH₂)ₙ—CHPh₂, or —CH₂CH₂CH(Ph)CH₂Ph (wherein n is 1 or 2);
Y represents S or O;
Z represents O, —CH₂—, NR₃, CHR₃ (wherein R₃ is hydrogen, lower alkyl having 1 to 5 carbon atoms, benzyl or phenethyl);
R₂ represents hydrogen, lower alkyl having 1 to 6 carbon atoms, cycloalkyl, dimethyl, or Ar″—(CH₂)ₚ— (wherein Ar″ is a phenyl substituted or unsubstituted with halogen or trifluoromethyl; or pyridinyl, imidazolyl or indolyl substituted or unsubstituted with carboxyl, amino, methanesulfonylamino or t-butoxycarbonyl, p is 0, 1, 2, 3 or 4.);
A represents O or —CH₂—; and
Ar represents 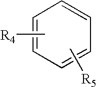
(wherein R′₄ and R₅ each independently are hydrogen, hydroxy, methoxy, nitro, cyano, benzyloxy, amino, methanesulfonylamino, halogen, lower alkyl having 1 to 5 carbon atoms, —NHCO₂CH₃, —NHC(═O)CH₃, trifluoromethyl, sulfamoyl, carboxyl, —OCH₂OCH₃, methoxycarbonyl); or pyridinyl, indolyl or imidazolyl substitituted or unsubstituted with carboxyl, amino, methanesulfonylamino, phenethylaminocarbonyl or t-butoxycarbonyl.

2. A pharmaceutical composition comprising the compound according to claim 1 or a pharmaceutically acceptable salt thereof as an active ingredient together with a pharmaceutically acceptable carrier.

3. The pharmaceutical composition according to claim 2, wherein the compound according to claim 1 or a pharmaceutically acceptable salt thereof as an active ingredient together with an acceptable carrier are present in an effective amount for preventing or treating pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, stomach-duodenal ulcer, inflammatory bowel disease or inflammatory diseases.

4. A method for preventing or treating pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, stomach-duodenal ulcer, inflammatory bowel disease or inflammatory diseases, wherein the method comprises administering a therapeutically effective amount of the compounds selected from the group consisting of compounds of formula I or pharmaceutically acceptable salts thereof.

5. The use of compounds selected from the group consisting of compounds of formula I or pharmaceutical acceptable salts thereof as an antagonist of vanilloid receptor.
